(19) United States
(12) Patent Application Publication
(10) Pub. No.: US 2025/0262247 A1
(43) Pub. Date: Aug. 21, 2025

(54) MICROGLIAL CELLS AS THERAPEUTIC TARGETS IN NEURODEVELOPMENTAL DISORDERS

(71) Applicant: The Regents of the University of California, Oakland, CA (US)

(72) Inventors: Alysson Renato Muotri, San Diego, CA (US); Pinar Mesci, Oceanside, CA (US)

(21) Appl. No.: 18/856,468

(22) PCT Filed: Apr. 20, 2023
(86) PCT No.: PCT/US2023/019217

§ 371 Date: Oct. 11, 2024

Related U.S. Application Data
(60) Provisional application No. 63/332,814, filed on Apr. 20, 2022.

(51) Int. Cl. A61K 35/30; A61K 31/427; A61P 25/00; C12N 5/079; G01N 33/50

(52) U.S. Cl. CPC A61K 35/30; A61K 31/427; A61P 25/00; C12N 5/0622; G01N 33/5023; G01N 33/5058; G01N 2800/2821; G01N 2800/285

(57) ABSTRACT

The disclosure provides methods and compositions related to microglial cell development and therapies in neuronal development.

### CROSS REFERENCE TO RELATED APPLICATIONS

This application claims priority under 35 U.S.C. § 119 from Provisional Application Ser. No. 63/332,814, filed Apr. 20, 2022, the disclosures of which are incorporated herein by reference.

### STATEMENT REGARDING GOVERNMENT SPONSORED RESEARCH

This invention was made with government support under MH107367 and N5105969, awarded by the National Institutes of Health (NIH). The government has certain rights in the invention.

### TECHNICAL FIELD

The disclosure provides for the discovery and development of therapies that can rescue neuronal defects caused by abnormal expression of neural-associated gene.

### BACKGROUND

Microglial cells originate from primitive hematopoiesis in the yolk sac during embryogenesis. Microglia are highly dynamic resident macrophages of the central nervous system (CNS) that can be beneficial contributors to tissue homeostasis or toxic contributors to brain pathology during inflammatory processes, such as in neurodegenerative diseases. Microglial cells are the first glial cells appearing in the brain, developing alongside with neurons during a critical period of neurodevelopment, coinciding with the beginning of synaptogenesis both in rodents and in humans. Growing evidence supports neuro-immune crosstalk that is crucial for brain development and function. Indeed, many studies have shown microglia can actively modulate neuronal activity and function by mechanisms such as synaptic pruning in rodents. However, due to limited in utero access, little is known about the microglial contribution to healthy human brain development. The few human studies mainly focused on microglial phenotypes and roles in late-onset pathological conditions of the aging brain, such as Alzheimer's disease. Thus, microglial involvement in human neurodevelopmental disorders remains largely unexplored.

### SUMMARY

The disclosure provides for the generation of microglial cells (MGL) from healthy hiPSCs and an established long-term (18 months) brain cortical organoid (BCO) and MGL co-culture systems. The addition of MGL to the BCO enhanced the expression of synaptic and neuronal activity genes, and also genes related to synaptic pruning and microglial signaling as revealed by single-cell RNA sequencing (scRNA-seq), suggesting a potential microglial involvement in neuronal connectivity. By comparing the transcriptional profile of MGL to human primary fetal microglia (FM), many Autism Spectrum Disorder (ASD)-related risk genes were expressed at similar levels between FM and MGL. Thus, it was hypothesized that human microglia, besides playing a role in healthy neurodevelopment, could also be involved in perturbed neurodevelopmental conditions such as ASD.

The disclosure provides a method for treating a disease or disorder caused by abnormal neuronal development in a subject in need thereof, comprising administering to the subject a therapeutically effective amount(s) of an agonist of CD11b and/or genetically normal microglial cells. In one embodiment, the subject is a female subject. In another embodiment, the subject is a male subject. In still another or further embodiment, the subject is less than 25 years of age. In still a further embodiment, the subject is less than 10 years of age. In still another or further embodiment of any of the foregoing, the agonist is ADH-503 or other leukadherin-1 CD11b agonist. In still another or further embodiment, the genetically normal microglial cells are derived from induced pluripotent stem cells (iPSCs). In another embodiment, the disease or disorder is Rett Syndrome. In yet another embodiment, the disease or disorder is selected from the group consisting of amyotrophic lateral sclerosis (ALS), Alzheimer's disease (AD), schizophrenia, autism spectrum disorders, diffuse leukoencephalopathy with spheroid formation, and frontal Lobar degeneration (FTLD). In another embodiment, the iPSCs are autologous to the subject. In still another embodiment, the iPSCs are from a subject having Rett syndrome and wherein the IPSCs are genetically modified to express MECP2. In another embodiment, the iPSCs are modified by CRISPR/Cas. In another embodiment, the subject has a deficit in normal phagocytosis and wherein the iPSCs are treated with a CD11b agonist prior to and concurrently with differentiation to microglial cells. In a further embodiment, the microglial cells are administered in combination with a CD11b agonist. In a further embodiment, the CD11b agonist is ADH-503.

The disclosure also provides a human-based neural drug screening platform for identifying therapeutic compounds that can ameliorate or rescue deleterious biological effect(s) resulting from abnormal expression or activity of genes in neuronal development disorders, comprising: (a) a two- and three-dimensional coculture of neurons and microglial cells, wherein (i) the neurons, (ii) the microglial cells or (iii) the neurons and microglial cells, have been differentiated from human stem cells that have mutation(s) affecting normal neuronal development; (b) contacting the coculture with a candidate agent; and (b) evaluating whether the candidate drug rescues or ameliorates deleterious gene expression; wherein if a candidate drug rescues and/or ameliorates one or more deleterious biological effects the candidate drug is a therapeutic compound. In another embodiment, the human stem cells are induced pluripotent stem cells (iPSCs). In a further embodiment, the iPSCs are obtained from a subject having a genetic neurological disease or disorder. In another embodiment, the candidate test agent is a small molecule drug. In a further embodiment, the small molecule drug effects CD11b expression and/or activity. In still a further embodiment, the small molecule drug mediates one or more activities selected from the group consisting of phagocytosis, cell adhesion and migration, restricts TLR and IFN-1 signaling, inhibits B cell activation, inhibit T cell activation, inhibits Th17 differentiation, suppresses CD maturation and function. In another embodiment, the small molecule agent promotes CD11b/CD18 dimerization to form CR3.

### DESCRIPTION OF DRAWINGS

FIG. 1A-L shows the absence of MECP2 leads to decreased cell viability, morphological and transcriptional changes involving dysregulations in cell migration, integrin and phagosome formation pathways in MGL. (A) Representative images of CTRL, KO, and KOR MGL stained with CD68 and MeCP2 antibodies. Scale bar 10 μm. (B) The percentage of cell viability was compared between different cell lines. Each data point shows the mean+/−SEM, as indicated. Significance was tested using a two-way ANOVA with the Tukey's multiple comparison test (*P=0.0304 and *P=0.0247 for day 24 and 29 respectively (three lines for CTRL MGL and KO and two isogenic rescue lines for KOR MGL, n=3 biological replicates for each sample at each time point). (C) Brightfield images of CTRL, KO and, KOR MGL at day 7 of differentiation, scale bar 200 μm. (D) Brightfield images of CTRL MGL with (right) or without (left) masks that marked round (in green), elongated (in blue) or cell clumps (in red), scale bar 200 μm. (E-H) Percentage of round cells, elongated cells and, cell clumps, respectively. (H) Area fold change compared to the control of CTRL, KO, and KOR. Significance was tested using a one-way ANOVA with the Tukey's multiple comparison test (*P=0.0323, **P=0.0022, and n.s. for E to G respectively for *P=0.0149 and **P=0.0021 for H (Each bar shows the mean+/−SEM, each dot represents one sample, two different isogenic pairs, two different clones for CTRL with three biological replicates per sample). (I) Heatmap showing the 39 differentially expressed genes (greater than 1.25-fold) between these two groups (KO n=5 from two independent cell lines, two to three biological replicates, CTRL n=5 from two independent cell lines, two to three biological replicates and KOR n=3 from two independent cell lines, one to two biological replicates P<0.05). Increasing fold changes compared to CTRL are marked in red while decreasing ones are in blue. (J) Top canonical pathways were obtained with 39 differentially expressed genes (DEG) using IPA, where significance was calculated by the right-tailed Fisher's Exact test (P<0.05). (K) The top network involving the majority of DEG obtained through IPA, genes that are downregulated are in green and upregulated are in red. (L) Top 5 diseases and biological functions were obtained with IPA.

FIG. 2A-I shows MeCP2 Chromatin-immunoprecipitation sequencing and proteomic assays on MGL. (A) Explanatory schematic of CTRL, KOR, and KO MGL regarding the role of MeCP2. (B) Venn Diagram of MeCP2 occupation peaks between CTRL and KOR. (C) Annotation distribution of MeCP2 target peaks. (D) Top Canonical Pathways for the promoter-TSS region obtained using IPA, where significance was calculated by the right-tailed Fisher's Exact test (P<0.05). (E) A table highlighting several MECP2 targets of interest in the genome. (F-G) Top 5 known and de novo motifs obtained with MeCP2 ChIP-seq in MGL. (H) LC-MS proteomic analyses conducted on MGL shows 480 overlapping candidates with MeCP2 ChIPseq in the promoter region. (I) Top Canonical Pathways of the intersecting candidates between proteomics and ChIP-seq obtained through IPA (P<0.05).

FIG. 3A-I shows KO MGL have increased glutamate, and ROS release decreased phagocytosis but the globally similar inflammatory response to LPS as CTRL and KOR MGL. (A) Glutamate release by MGL. Bars represent mean+/−SEM. Significance was tested by one-way ANOVA with Tukey's multiple comparison test (*P=0.0329, two different KO lines, one rescue line and two independent CTRL MGL lines with two clones). (B) Measurement of reactive oxygen species (ROS) released in the conditioned media by MGL. One-way ANOVA with Tukey's multiple comparison test was used to assess the significance (****P<0.0001). Bars represent mean+/−SEM. Two independent cell lines for CTRL and KO MGL, and one isogenic rescue line with three biological replicates per sample were used. (C) Migration assays using transwell chambers using ATP or CXC3L1. Significance was tested by one-way ANOVA with Tukey's multiple comparison test (*P=0.0261), two independent cell lines for CTRL and KO MGL, and one isogenic rescue line with three biological replicates per sample. (D) Heatmap showing the means for each cytokine released in the conditioned media for a given genotype (blue to red, low to high concentration in the media in pg/ml), three independent CTRL and KO MGL lines and two isogenic rescue lines were used. (E-F) MIP-1alpha and GM-CSF released in the conditioned media by MGL was measured respectively (in pg/ml). Significance was tested by one-way ANOVA with Tukey's multiple comparison test (****P<0.0001 and *P=0.0179, three independent CTRL and KO MGL lines and two isogenic rescue lines were used). Bars represent mean+/−SD. (G) Brightfield images of CTRL, KO, and KOR MGL engulfing zymosan particles. Once engulfed, the zymosan particles fluoresce in red, scale bar 200 μm. Blue is a live nuclear stain (Nucleo Blue). (H) Phagocytosis percentage of zymosan particles compared to CTRL MGL. Significance was tested by One-way ANOVA with Tukey's multiple comparison test (****P<0.0001 and *P=0.0289, two different isogenic KO/KOR pairs, at least four biological replicates each, two independent CTRL MGL lines with three clones). Bars represent mean+/−SEM. Each dot represents one sample. (I) Phagocytosis of pHrodo-conjugated brain organoid-derived synaptosomes-enriched fractions measured as red fluorescent area normalized to hour=0 and to CTRL baseline phagocytosis using incucyte. Significance was tested by one ANOVA (*P<0.001) using one control and one KO isogenic pair with 5 biological replicates each. Bars represent mean+/−SEM. (*P=0.0184 and *P=0.0269 respectively).

FIG. 4A-L shows KO^{R} MGL elicits transcriptional changes in KO BCO associated with synaptogenesis and rescues the synaptic defects of KO neurons in long-term co-culture experiments. (A) Brightfield images of neuron-MGL (labeled with long-term stable membrane stain PKH26 in red) co-culture on day 2 and 50 of neuronal differentiation, scale bar 400 μm. (B) Representative images of co-cultures neurons (stained with MAP2) and MGL (stained with CD68) for 2 months. The post-synapse is stained with HOMER1. The last panel shows a magnification of the square area shown. Note that MGL is nearby of MAP2+ neurons and synapses, scale bar 50 μm. (C) Representative images of synaptic puncta co-localization with or without CTRL, KO or KOR MGL, scale bar 20 μm. (D) Quantification of the number of synaptic puncta (KO vs. CTRL neurons without MGL *P=0.0183; CTRL neurons with CTRL MGL vs. CTRL neurons with KO MGL **P=0.0029; KO neurons without MGL vs. KO neurons with CTRL MGL *P=0.0299; KO neurons with CTRL MGL vs. KO neurons with KO MGL *P=0.0114, one isogenic KO/KOR pair, and one CTRL MGL line was used, synaptic puncta from 10 neurons were counted, the experiment was run in two independent batches). (E) Quantification of the number of co-localized synaptic puncta (CM=conditioned media from CTRL or KO MGL) (*P=0.0482 one isogenic KO/KOR pair and one CTRL MGL line was used, synaptic puncta from 10 neurons were counted). (F) Quantification of the number of co-localized synaptic puncta without healthy human primary fibroblasts (Fibro), (**P=0.0084, one isogenic KO/KOR pair, and one CTRL MGL line was used, synaptic puncta from 10-15 neurons were counted). (G) Brightfield images of spheroids co-cultured with MGL (in red) before and after plating on MEA plates, scale bar 400 μm. (H) Graph showing the spike rate compared to CTRL spheroids without any MGL, recorded in five minutes emerging from spheroids with or without CTRL or KOR MGL. **P=0.0021, *P=0.0150, n.s. not significant, one isogenic rescue line, and two different CTRL and KO lines were used, two to three biological replicates per genotype were used. Significance is assessed by one-way ANOVA with Tukey's multiple comparison test for the experiments in D, E, F, and H. Number of neurons counted for synaptic puncta is represented by one data point in each graph. All synaptic puncta quantification was calculated as a percentage compared to CTRL neurons. Bars represent mean+/−SEM.

FIG. 5A-H show ADH-503 rescues phagocytosis in KO MGL, improves disease score and survival. (A) Schematic of the compounds used in the screening; agonist compounds are in red and inhibitors in blue. (B) Drug screening using phagocytosis assay. Phagocytosis percentage of zymosan particles compared to CTRL MGL. Significance was tested by two-way ANOVA (****P<0.001, *P=0.0109, **P=0.0041), experiment run in two batches, n=12 to 24 biological replicates. Bars represent mean+/−SEM. (C) MECP2 binding events to ITGAM (CD11b) promoter, baseline binding events detected (untreated) or at the promoter region of ITGAM. Significance was tested by two-way ANOVA followed by Sidak multiple comparison test (***P=0.0004, *P=0.0236, CTRL vs KO ITGAM-190 bp **P=0.0013, untreated KOR vs ITGAM-190 bp **P=0.0019), n=3 biological replicates. Bars represent mean+/−SEM. (D) Peaks detected in the promoter region of ITGAM in CTRL and KOR MGL by MeCP2 ChIP-seq. (E) Schematic of ADH-503 treatment of MeCP2-KO male mice. (F) Average total symptom score was calculated in each group at indicated time points. MeCP2-KO mice treated with ADH-503 showed a significant reduction in total score compared with vehicle-treated MeCP2-KO mice. N=KO-vehicle: 11, KO-ADH-503:10. Significance was tested with Student's t-test, week 8; ***P=0.0004, week 9, ***P=0.0007. (G) Kaplan-Meier survival curves. MeCP2-KO mice treated with ADH-503 survived significantly longer than non-treated control MeCP2-KO mice (*P=0.0244; log-rank test), with median survival of 63.5 days (for control mice) and 91 days (for ADH-503-treated mice). N=KO: 20, KO-ADH-503:10. (H) Cryosections of WT or MeCP2-KO mouse brains treated with vehicle or ADH-503 stained with NeuN (scale bar 100 μm, on the left) and measurement of soma size was plotted on a bar graph. Significance was tested by one-way ANOVA with Tukey's multiple comparison test (**P=0.0078, ***P=0.0004), n=6 mice/group.

FIG. 6A-G shows characterization of MGL compared to FM. (A) Protocol for generating MGL from iPSCs. (B) Representative images of hiPSC-derived CTRL MGL after staining with classical microglial markers, CD68, CX3CR1, TREM2, IBA1, PU.1, CD11b, and P2YR12. Scale bar 20 μm. (C) Sample correlation analysis. The analysis was based on regularized log-transformed data and calculated from RNA-seq data. (D) Sample variation based on 2D Principal Component Analysis (PCA) Analysis was based on normalized read counts of per-sample RNA-seq data (n=3 samples for FM, n=4 for MGL from two different controls and two biological replicates, n=5 iPSC-derived control neurons from two different cell lines with two biological replicates per sample, one to two different clones and, n=2 iPSC-derived control astrocytes). (E) Heatmap of MGL and fetal microglia gene expression (FM). (F) Heatmap comparing MGL and fetal microglia gene expression (FM). The MGL express hematopoietic stem cell (HSC), primitive hematopoietic progenitor cell (HPC), erythromyeloid progenitor (EMPs) and microglial markers at comparable levels to FM. (G) Comparison of gene expression (TPM) of several autism spectrum disorders (ASD)-related genes between MGL and FM showing similar levels of expression. Truncated violin plots show the distribution of samples for each ASD-related gene studied. Significance was assessed using a two-way ANOVA test followed by Sidak's multiple comparison test, ****P<0.0001, **P<0.01, n.s. not significant (n=3 samples for FM, n=4 for MGL from two different controls and two biological replicates)

FIG. 7A-K shows characterization of hiPSC-derived CTRL, KO, and KOR microglia-like cells (MGL) at the transcriptional level. (A) Schematic of the CRISPR/Cas9 strategy used to generate isogenic KO lines. The guide RNA is highlighted with a blue line and the PAM cutting region in the red triangle. (B) Immunostainings for MECP2 in CTRL, KO, and KOR MGL, the MeCP2 integrated density was normalized to cell number. Significance was tested by one-way ANOVA with Tukey's multiple comparison test. Bars represent mean+/−SEM. Each dot represents one sample. **P=0.0032, *P=0.0424, three lines for CTRL MGL and KO and two isogenic rescue lines for KOR MGL, n=3 biological replicates for CTRL and KO MGL and n=2 biological replicates for KOR MGL were used. (C) Brightfield images of CTRL, KO, and KOR MGL at day 2, 4, and 6 of microglial differentiation, scale bar 400 μm. (D) Volcano plot showing differences between KO VS. CTRL and KOR MGL. 39 genes were differentially expressed (greater than 1.25-fold) between these two groups (KO n=5 from two independent cell lines, two to three biological replicates, CTRL n=5 from two independent cell lines, two to three biological replicates and KOR n=3 from two independent cell lines, one to two biological replicates P<0.05). Several integrins and collagen genes are highlighted. (E) The phagosome formation pathway, a pathway among the activated pathways, was generated using the IPA software. (F) Neuroinflammation signaling pathway, another pathway among the activated pathways, was generated using the IPA software. The pathway includes genes such as TREM2, TYROBP, or NOX. (G) Multidimensional scaling (MDS) showing expression differences between CTRL, KO, and KOR MGL. (H) Sample correlation heatmap between CTRL, KO, and KOR MGL. (I) Top Canonical Pathways obtained from MGL bulk RNA sequencing, generated by Ingenuity Pathway Analysis Software. (J) Gene expression heatmap (1.5 fold-change, P<0.05). (K) The data is represented as violin plots where several groups of genes are highlighted, notably microglial markers, complement system, cathepsins, chemokine signaling, SIGLECs, and ER-Phagosome Formation (1.5 fold-change, P<0.05).

FIG. 8A-G shows MECP2 ChIP-Seq and proteomic analyses performed on MGL. (A) Transcription Start Sites (TSS) plot of KOR and CTRL MGL MECP2 ChIP seq samples. (B) Autocorrelation plot for the input, KOR, and CTRL MGL MECP2 ChIP-seq samples. (C) Significant Peaks for MECP2 occupation in the promoter-TSS region present in both KOR and CTRL samples. (D) Top Canonical Pathways generated from the proteomics data of KO MGL compared to CTRL and KOR samples using IPA. (E-F) Top causal networks and Top diseases and bio functions generated from the proteomics data of KO MGL compared to CTRL and KOR samples using IPA. (G) Venn Diagram of overlap between proteins and genes found in RNA-seq and NanoString myeloid innate immunity panel.

FIG. 9A-C shows no overt inflammation signs in KO MGL compared to CTRL. (A) The release of Eotaxin, Eotaxin-3, IL8, VEGF, IP-10, MCP-1, MDC, IL-13, TNFalpha, MIP-1beta, TARC, IL-10, IL6, IL12/IL23p40, IL-15, IL-1beta, IL16, IL-17A, IL-1alpha, IL-12p70, IL-5, IL-7, TNF beta and IL-2 and was measured in the conditioned media by MGL (in pg/ml). Note that most of the cytokines increase upon activation with LPS. Significance was tested by One-way ANOVA with Tukey's multiple comparison test. Bars represent mean+/−SD. Each dot represents one sample, three independent CTRL and KO MGL lines and two isogenic rescue lines were used. (B) Schematic of synaptosomes phagocytosis assay. (C) Phagocytosis percentage of zymosan particles compared to CTRL MGL with or without Cytochalasin D, an inhibitor of phagocytosis. Significance was tested by One-way ANOVA with Tukey's multiple comparison test (****P<0.0001, **P=0.0013 and *P=0.0235, one KO patient cell line and two different CTRL MGL and two clones were used with three to six biological replicates). Bars represent mean+/−SEM.

FIG. 10A-O shows KOR MGL elicits transcriptional changes in KO BCO associated with synaptogenesis and rescues the synaptic defects of KO neurons in long-term co-culture experiments. (A) Representative images of neuron-microglia co-cultured for 8 weeks, stained HOMER1 (in green), MAP2 (in white), CD68 (in orange), and DAPI (in blue). Scale bars 50 μm and 20 μm (upper and lower panel for each condition, respectively). (B) Schematic of 2D neuron-MGL co-cultures. (C) Comparison of gene expression (TPM) of Colony stimulating factor 1 receptor (CSF1R) that binds both IL34 and CSF1 in hiPSC-derived neurons vs MGL. Bars represent mean+/−SEM. Significance was assessed using a Student's t-test, ****P<0.0001, (n=7 biological replicates samples for neurons, n=6 for MGL two different controls). (D) Quantification of the number of synaptic puncta (KO vs. CTRL neurons without MGL *P=0.0183; CTRL neurons with CTRL MGL vs. CTRL neurons with KO MGL **P=0.0029; KO neurons without MGL vs. KO neurons with CTRL MGL *P=0.0299; KO neurons with CTRL MGL vs. KO neurons with KO MGL *P=0.0114, one isogenic KO/KOR pair, and one CTRL MGL line was used, synaptic puncta from 10 neurons were counted, the experiment was run in two independent batches). (E) Heatmap table showing the percentages of increase (in green) or decrease (in red) with different combinations of co-culture with MGL. (F) Representative images of neurons co-cultured with either KO or CTRL MGL, stained with MAP2, scale bar 50 μm. (G) Neurometric analysis of neurons co-cultured with microglial cells showing the number of branching points, total neurite length, mean length per branch, minimum branch length, maximum branch length, and soma size in μm. Data was analyzed by one-way ANOVA with Tukey's multiple comparison test for all the experiments (*P<0.05, ***P<0.001, ****P<0.0001, n.s not significant). Bars represent mean+/−SEM, one isogenic KO/KOR pair, and one CTRL MGL line was used, neurites from 10 neurons were analyzed per sample. (H) Schematic of the experimental design using conditioned media from MGL. (I) Schematic of the experimental design using primary control fibroblasts. (J) Representative images of synaptic puncta co-localization with or without CTRL MGL conditioned media (CM), HOMER1 is in green, VGLUT1 in red, and MAP2 in white, scale bar 20 μm. (K) Representative images of synaptic puncta co-localization with or without CTRL primary fibroblasts, HOMER1 is in green, VGLUT1 in red, and MAP2 in white, scale bar 20 μm. (L) Schematic of the 3D spheroid-MGL co-culture. (M) Brightfield images of spheroids co-cultured with MGL (in red) before and after plating on MEA plates, respectively, scale bar 400 μm. Arrows point to microglia-like cells, stained with the membrane dye PKH26. (N) Raster plots of neurospheres recorded for 5 minutes with or without MGL. (O) Quantitative measurements of the number of active electrodes, number of spikes, number of bursts, burst duration, and number of spikes per bursts and were analyzed during neuronal activity recording using Axion Biosystems. Significance is assessed by one-way ANOVA with Tukey's multiple comparison test; each dot represents a biological replicate. CTRL spheroids vs. CTRL spheroids with CTRL MGL, *P=0.0106, CTRL spheroids with CTRL MGL vs. KO spheroids without MGL, **P=0.0025, one isogenic rescue line, and two different CTRL and KO lines, two to three biological replicates per genotype were used, n.s. not significant.

### DETAILED DESCRIPTION

As used herein and in the appended claims, the singular forms “a,” “an,” and “the” include plural referents unless the context clearly dictates otherwise. Thus, for example, reference to “a prodrug” includes a plurality of such prodrugs and reference to “the agent” includes reference to one or more agents and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this invention is not limited to the particular methodology, protocols, and reagents, etc., described herein and as such may vary. The terminology used herein is for the purpose of describing particular embodiments only and is not intended to limit the scope of the invention, which is defined solely by the claims.

Other than in the operating examples, or where otherwise indicated, all numbers expressing quantities of ingredients or reaction conditions used herein should be understood as modified in all instances by the term “about.” The term “about” when used to described the invention, in connection with percentages means ±10%, 5%, or typically 18.

Microglia are tissue-specific brain macrophages. Microglia perform several roles in the development and maintenance of the central nervous system (CNS). Microglia arise from primitive CD45⁺CX3CR1⁻ myeloid progenitors in the yolk sac that differentiate to CD45⁺CX3CR1⁺ microglial progenitors and invade the developing brain before the emergence of definitive hematopoiesis. In a healthy, adult brain with an intact blood brain barrier, microglia persist as a long-lived, self-sustained population that is not replenished by circulating bone marrow-derived cells. Highly branched microglia cells, defined as “resting”, are in reality highly active as their processes continuously move to examine the brain for homeostatic disruptions.

Microglia use phagocytosis to eliminate pathogens and/or damaged or dead cells, and remove toxic molecules, cellular debris, and/or protein deposits, thus attenuating inflammation and promoting tissue regeneration and repair. During development, microglia promote migration and differentiation of neural progenitors, neurogenesis, and oligodendrogenesis, and regulate synaptogenesis and synaptic plasticity. Microglia also contribute to pathological brain inflammation and disruption of the blood-brain barrier by releasing cytokines and neurotoxic molecules. Dysfunctional microglia have been linked to amyotrophic lateral sclerosis (ALS) and Alzheimer's disease (AD).

The term “defined” or “fully-defined,” when used in relation to a medium, an extracellular matrix, or a culture condition, refers to a medium, an extracellular matrix, or a culture condition in which the chemical composition and amounts of approximately all the components are known. For example, a defined medium does not contain undefined factors such as in fetal bovine serum, bovine serum albumin or human serum albumin. Generally, a defined medium comprises a basal media (e.g., Dulbecco's Modified Eagle's Medium (DMEM), F12, or Roswell Park Memorial Institute Medium (RPMI) 1640, containing amino acids, vitamins, inorganic salts, buffers, antioxidants, and energy sources) which is supplemented with recombinant albumin, chemically defined lipids, and recombinant insulin. An example of a fully defined medium is Essential 8™ medium.

A disease or disorder associated with microglia abnormalities or a neurological disease or disorder involving microglia include, e.g., disease and disorders having phagocytosis dysfunctions. The term “neurodegenerative diseases or disorders” and “neurological diseases and disorders” encompass a disease or disorder in which the peripheral nervous system or the central nervous system is principally involved. The compounds, compositions, and methods provided herein may be used in the treatment of neurological or neurodegenerative diseases and disorders. As used herein, the terms “neurodegenerative disease”, “neurodegenerative disorder”, “neurological disease”, and “neurological disorder” are used interchangeably. Examples of diseases or disorders of the disclosure includes, but are not limited to, Alzheimer's disease (AD), Parkinson's, ischemia, frontotemporal dementia, schizophrenia, multiple sclerosis, retinal degeneration, infections, ischarmia, aging, autism, bipolar disorder, nasu-hakola, autism spectrum disorders, aicardi-goutieres syndrome, amyotrophic lateral sclerosis (ALS), diffuse leukoencephalopathy with spheroid formation, and frontal Lobar degeneration (FTLD), Rett syndrome and other neurodevelopment disorders. Further disease and disorders can include, but are not limited to, chronic neurological diseases such as diabetic peripheral neuropathy (including third nerve palsy, mononeuropathy, mononeuropathy multiplex, diabetic amyotrophy, autonomic neuropathy and thoracoabdominal neuropathy), age-related memory loss, senility, age-related dementia, Pick's disease, diffuse Lewy body disease, progressive supranuclear palsy (Steel-Richardson syndrome), multisystem degeneration (Shy-Drager syndrome), degenerative ataxias, cortical basal degeneration, ALS-Parkinson's-Dementia complex of Guam, subacute sclerosing panencephalitis, Huntington's disease, synucleinopathies, primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Wemicke-Korsakoffs related dementia (alcohol induced dementia), Werdnig-Hoffmann disease, Kugelberg-Welander disease, Tay-Sach's disease, Sandhoff disease, familial spastic disease, Wohifart-Kugelberg-Welander disease, spastic paraparesis, progressive multifocal leukoencephalopathy, and prion diseases (including Creutzfeldt-Jakob, Gerstmann-Straussler-Scheinker disease, Kuru and fatal familial insomnia).

“Feeder-free” or “feeder-independent” is used herein to refer to a culture supplemented with cytokines and growth factors (e.g., TGFbeta, bFGF, LIF, analogs or mimetics thereof) as a replacement for the feeder cell layer. Thus, “feeder-free” or feeder-independent culture systems and media may be used to culture and maintain pluripotent cells in an undifferentiated and proliferative state. In some cases, feeder-free cultures utilize an animal-based matrix (e.g. MATRIGEL™) or are grown on a substrate such as fibronectin, collagen, or vitronectin. These approaches allow human stem cells to remain in an essentially undifferentiated state without the need for mouse fibroblast “feeder layers.”

“Feeder layers” are defined herein as a coating layer of cells such as on the bottom of a culture dish. The feeder cells can release nutrients into the culture medium and provide a surface to which other cells, such as pluripotent stem cells, can attach.

“Induced pluripotent stem cells (iPSCs)” are cells generated by reprogramming a somatic cell by expressing or inducing expression of a combination of factors (herein referred to as reprogramming factors). iPSCs can be generated using fetal, postnatal, newborn, juvenile, or adult somatic cells (e.g., fibroblasts are commonly used). In certain embodiments, factors that can be used to reprogram somatic cells to pluripotent stem cells include, for example, Oct4 (sometimes referred to as Oct 3/4), Sox2, c-Myc, Klf4, Nanog, and Lin28. In some embodiments, somatic cells are reprogrammed by expressing at least two reprogramming factors, at least three reprogramming factors, or four reprogramming factors to reprogram a somatic cell to a pluripotent stem cell.

“Prevent” or “prevention” refers to prophylactic or preventative action that prevent and/or slow the development of a pathologic disease or disorder. In certain embodiments, a disease or disorder is successfully prevented according to the methods provided herein if the subject develops, transiently or permanently, fewer or less severe symptoms associated with the disease or disorder, or a later onset of symptoms associated with the disease or disorder, than a subject who has not been subject to the methods of the invention.

By “subject” or “patient” is meant any subject, particularly a mammalian subject, for whom diagnosis, prognosis, or therapy is desired. Mammalian subjects include humans, domestic animals, farm animals, sports animals, and zoo animals including, e.g., humans, non-human primates, dogs, cats, guinea pigs, rabbits, rats, mice, horses, cattle, pigs, and so on. In some embodiments, the subject is a human subject.

As used herein the phrase “substantially pure” refers to a population of cells wherein at least 90%, 95%, 98%, 99% or more of the cells have a desired phenotype. In all embodiments that refer to a “substantially pure” cell population, alternative embodiments in which the cell populations have a lower or higher level of purity are also contemplated.

A “three-dimensional culture” refers to an artificially-created environment in which biological cells are permitted to grow or interact with their surroundings in all three dimensions. A 3D culture can be grown in various cell culture containers such as bioreactors. In contrast, a “two-dimensional culture” refers to a cell culture such as a monolayer on an adherent surface.

The term “treating” or “treatment” or “to treat” refer to therapeutic actions that cure, slow down, lessen symptoms of, and/or halt progression of a pathologic disease or disorder. In certain embodiments, a subject is “treated” for a disease or disorder if the subject shows, e.g., total, partial, permanent, or transient, alleviation or elimination of any symptom associated with the disease or disorder.

The disclosure provides direct evidence that MGL is causally involved in human synaptogenesis and neural network establishment. Specifically, the disclosure shows that human MGL promote enhanced neuronal synapse formation and neuronal activity through physical contact using long-term neural cultures. Not only has microglial identity been shown to be defined by their neuronal environment, but the results suggest that the optimal function and maturation of neuronal cells are impacted by microglial cells in a human model; revealing a previously under-appreciated non-cell autonomous role of microglia on neuronal connectivity and synapse formation.

To further understand the contribution of human MGL to disease states, similarities between fetal microglia (FM) and microglial cells (MGLs) regarding the levels of ASD-related gene expression were examined. The disclosure demonstrates morphological alterations and a latency period in the MGL generation from knockout (KO) samples. Myeloid-specific transcriptional factor, PU.1, was identified as one of the top 5 known binding motif sites for microglial MECP2 in a ChIP-sequencing experiment due to its direct interaction with MECP2, demonstrating that MECP2 might also play a role during primitive hematopoiesis.

The multi-omics of the disclosure repeatedly showed that several microglial functions such as phagocytosis, migration, and neuroinflammation signaling pathways could be altered. KO MGL revealed similarities to the findings in Rett Syndrome (RTT) mouse models, such as increased glutamate release into the media, and defective phagocytosis. KO MGL also had an increased ROS production, and impaired chemotaxis to fractalkine and response to LPS stimulation similar to control MGL, suggesting their ability to react to an immune stimulus remains globally intact.

The disclosure established 2D long-term co-culture systems and showed that CTRL/KO^{R} MGL functionally rescued the synaptogenesis defect of MeCP2-lacking neurons. In contrast to mouse studies, human MGL conditioned media alone did not impact co-localized synaptic puncta (CSP) number, suggesting that molecules secreted from KO MGL are not sufficient to elicit changes in CSP. Instead, the complementary data showed that signaling pathways such as integrin, chemokine, actin cytoskeleton, and complement signaling, explains how CTRL MGL rescue the synaptic defects in MECP2 KO neurons. These data also indicate that adding KO MGL could have a deleterious effect on synaptogenesis.

Phagocytosis is a complex receptor-mediated process that requires three main parts; “find-me,” “eat-me” and “digest-me,” each of which in turn is regulated by different receptors, molecules and signaling pathways. Although recent studies have begun to focus on microglial phagocytosis in adult neurological disorders, the contribution of microglial engulfment to human neurodevelopmental conditions has been largely unexplored. The disclosure demonstrates alterations at each step of the phagocytosis process in KO MGL. Interestingly, microglial phagocytosis has been shown to be important in mouse models of Rett Syndrome. By using phagocytosis as a therapeutic target, several compounds known to be agonists or inhibitors of different complement receptors or integrins were tested for their involvement in the process of phagocytosis. One compound, ADH-503, was able to restore the phagocytic function in KO MGL. ADH-503 is an agonist of CD11b, which, together with CD18, forms CR3, and interestingly, the disclosure shows that MECP2 occupies the promoter of ITGAM, the gene encoding for CD11b. ADH-503 is a small molecule that can activate CD11b, which was previously developed and used as a treatment of pancreatic cancer and is currently in phase 1 of clinical trials. Daily treatment of MeCP2-KO mice, whose microglial phagocytosis is also altered, with ADH-503 led to a significant improvement of symptom progression and increased survival. Moreover, despite the treatment starting at 3 weeks, hence after disease onset, the treatment is still able to significantly improve the progression of the disease. Apart from its role on rescuing neuronal soma size, ADH503-treatment might improve the overall symptoms by acting on peripheral macrophages/other myeloid cells that also express CD11b and were shown to have altered phenotypes in RTT mouse models.

The data revealed that MeCP2 is required for microglial functions such as phagocytosis, likely through CD11b, and supports that MGL plays a role in human neurodevelopment, notably in synapse formation. The disclosure also demonstrates differences with rodent models regarding inflammation and the role of secreted factors on neurotoxicity, highlighting the importance of using a human model to understand the disease-relevant mechanisms. Improving the generation of human iPSC-derived microglial surrogates to replace the unhealthy host microglia represent a cell-based therapy.

The data presented herein establish the groundwork to identify molecules involved in human neuro-immune interactions during development. The results provide clinical evidence that human microglial cells can be used as therapeutic targets that can lead to novel and efficient treatments for uncurable neurological disorders. Given that microglial cells are immune cells of the CNS, implicated in all neurological conditions, focusing on studying microglial disease phenotypes and performing drug screenings specifically to rescue altered microglial phenotypes paves the way to innovative treatments for neurodevelopmental but also broader neurological conditions.

The disclosure also provides evidence that MGL are involved in human synaptogenesis and neural network establishment. Specifically, human MGL are shown to promote the expression of genes involved in synaptic pruning, neuronal plasticity/activity, accompanied by an enhanced neuronal synapse formation and neuronal activity through physical contact using long-term neural cultures. Microglial identity has been shown to be heavily defined by their neuronal environment. In addition, the optimal function and maturation of neuronal cells are impacted by microglial cells in a human model; revealing a previously under-appreciated non-cell autonomous role of microglia on neuronal connectivity and synapse formation.

The results draw attention to the importance of human microglia during long-term neurodevelopment and neuronal functional maturation. By analyzing the gene expression changes in MGL-BCO 3D co-cultures at different time points, MGL were demonstrated to have a dynamic impact on brain cortical-organoids (BCO) depending on the developmental time point, therefore, proving their capability of adapting to environmental changes during neuronal development and functional maturation.

The disclosure also demonstrates that one or more compounds/agents may be tested to determine if the compound/agent has effects that may be beneficial for the treatment of a neurological disease or disorder. Based on the effects of the compound/agent on the functional activity or a disease marker, one may then be able to determine if the compound is useful for the treatment of a disease or disorder. In some embodiments, the cells are derived from iPS cells from a subject that has a disease (e.g., a genetic disease or a disease with a genetic component or risk factor) such as a neurological or neurodegenerative disease (e.g., Rett syndrome, autism, epilepsy, ADHD, schizophrenia, bipolar disorder, etc.). Co-cultures of microglia and/or cortical cells derived from the iPSCs are then contacted with the test agent. For example, one or more test agents may be added at varying concentrations to the co-culture medium. Depending upon the neurological disease or disorder, an agent that promotes or in some instances decreases (depending upon the disease) the expression of a polypeptide of interest can be considered useful as a therapeutic. In another embodiment, the activity or function of a microglia or cortical neuronal cell of an organoid is compared in the presence and the absence of a test agent.

Agents that can be tested can be identified from large libraries of natural product or synthetic (or semi-synthetic) extracts or chemical libraries or from polypeptide or nucleic acid libraries, according to methods known in the art. Those skilled in the art of drug discovery and development will understand that the precise source of test agents or compounds is not critical to the screening procedure(s). Agents used in screens may include those known as therapeutics for the treatment of neurological conditions. Alternatively, virtually any number of unknown agents or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

The assays to determine biological effects on the cells may include survival assays, microglia phagocytosis assays, calcium assays, calcium flux assays, synaptic pruning assays, signal transduction assays.

The assays may be performed in a high-throughput manner. For example, the co-cultures can be positioned or placed on a culture dish, flask, roller bottle or plate (e.g., a multi-well culture plate comprising 8, 16, 32, 64, 96, 384 and 1536 wells). Libraries that can be screened include, for example, small molecule libraries, siRNA libraries, antibody libraries, and the like. The screening platform may be automated, such as robotic automation. The culturing platform may comprise an automated cell washer and high content imager.

The disclosure also provides composition (including pharmaceutical compositions) comprising microglia cells and/or small molecule therapeutics useful to modulating phagocytosis. Such composition are useful for treating neurological disease and disorders. The disclosure also provides composition comprising MGL cells that can be administered in a pharmaceutical composition. In one embodiment, the MGL cells are derived from iPSCs that are autologous to the subject. In other embodiments, the cells are allogeneic to the subject.

Any reference to a drug herein also encompasses all of the pharmaceutically acceptable isomers (e.g., stereoisomers), solvates, hydrates, polymorphs, salts, and prodrugs (e.g., esters and phosphates). Accordingly, reference to ADH-503 includes other leukadherin-1 compounds or other CD11b agonists.

The disclosure further provides for a pharmaceutical composition comprising therapeutic compound(s) and compositions as disclosed herein, salts of the foregoing and combinations thereof disclosed herein. Any of a variety of art-known methods can be used to administer a therapeutic compound(s). For example, administration can be parenterally, intrathecally, cerebrally and the like, by injection or by gradual infusion over time. The therapeutic compositions and/or compound(s) alone or with the other therapeutic agents can be administered intravenously, intraperitoneally, intracranially, and/or intrathecally.

A pharmaceutical composition comprising cells or therapeutic compound(s) of the disclosure can be in a form suitable for administration to a subject using carriers, excipients, diluents and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, chelating agents, and inert gases. Other pharmaceutically acceptable carriers include aqueous solutions, non-toxic excipients, including salts, preservatives, buffers and the like, as described, for instance, in Remington's Pharmaceutical Sciences, 15th ed., Easton: Mack Publishing Co., 1405-1412, 1461-1487 (1975), and The National Formulary XIV., 14th ed., Washington: American Pharmaceutical Association (1975), the contents of which are hereby incorporated by reference. The pH and exact concentration of the various components of the pharmaceutical composition are adjusted according to routine skills in the art. See Goodman and Gilman's, The Pharmacological Basis for Therapeutics (7th ed.).

The disclosure further provides for a pharmaceutical composition that can be administered in a convenient manner, such as by injection (subcutaneous, intravenous, intracranial, intraspinal etc.), oral administration, inhalation, transdermal application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

A “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

The pharmaceutical composition can be orally administered, for example, in the case of small molecule agents with an inert diluent or an assimilable edible carrier. The pharmaceutical composition and other ingredients can also be enclosed in a hard or soft-shell gelatin capsule, compressed into tablets, or incorporated directly into the individual's diet. For oral therapeutic administration, the pharmaceutical composition can be incorporated with excipients and used in the form of ingestible tablets, buccal tablets, troches, capsules, elixirs, suspensions, syrups, wafers, chewable tablets, gummies, and the like. Such compositions and preparations should contain at least 18 by weight of active compound. The percentage of the compositions and preparations can, of course, be varied and can conveniently be between about 5% to about 80% of the weight of the unit.

The tablets, troches, pills, capsules, and the like can also contain the following: a binder, such as gum gragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic/biocompatible in the amounts employed. In addition, the pharmaceutical composition can be incorporated into sustained-release preparations and formulations.

In further embodiments, the disclosure further provides that oral pharmaceutical formulations comprising therapeutic compound(s) (e.g., ADH-503 and the like) disclosed herein may have an enteric coating. As used herein “enteric coating”, is a material, a polymer material or materials which encase the medicament core (e.g., prodrug the disclosure). Typically, a substantial amount or all of the enteric coating material is dissolved before the medicament or therapeutically active agent is released from the dosage form, so as to achieve delayed dissolution of the medicament core. A suitable pH-sensitive polymer is one which will dissolve in intestinal juices at a higher pH level (pH greater than 6), such as within the small intestine and therefore permit release of the pharmacologically active substance in the regions of the small intestine and not in the upper portion of the GI tract, such as the stomach. An “enterically coated” drug or tablet refers to a drug or tablet that is coated with a substance—i.e., with an “enteric coating”—that remains intact in the stomach but dissolves and releases the drug once the small intestine is reached.

The coating material is selected such that the therapeutically active agent will be released when the dosage form reaches the small intestine or a region in which the pH is greater than pH 6. The coating may be a pH-sensitive material, which remains intact in the lower pH environs of the stomach, but which disintegrate or dissolve at a more neutral pH commonly found in the small intestine of the patient. For example, the enteric coating material begins to dissolve in an aqueous solution at pH between about 6 to about 7.4.

Enteric coatings include, but are not limited to, beeswax and glyceryl monostearate; beeswax, shellac and cellulose; and cetyl alcohol, mastic and shellac, as well as shellac and stearic acid (U.S. Pat. No. 2,809,918); polyvinyl acetate and ethyl cellulose (U.S. Pat. No. 3,835,221); and neutral copolymer of polymethacrylic acid esters (Eudragit L30D) (F. W. Goodhart et al., Pharm. Tech., pp. 64-71, April 1984); copolymers of methacrylic acid and methacrylic acid methylester (Eudragits), or a neutral copolymer of polymethacrylic acid esters containing metallic stearates (Mehta et al., U.S. Pat. Nos. 4,728,512 and 4,794,001). Such coatings comprise mixtures of fats and fatty acids, shellac and shellac derivatives and the cellulose acid phthalates, e.g., those having a free carboxyl content. See, Remington's at page 1590, and Zeitova et al. (U.S. Pat. No. 4,432,966), for descriptions of suitable enteric coating compositions.

In another embodiment, the therapeutic composition can be formulated as an immediate release formulation such that it is delivered in the upper gastrointestinal tract. In still another formulation the therapeutic composition can comprise a gastroretentive formulation comprising, e.g., carbopol or some other hydrophilic polymer.

Preparations for parenteral administration of a composition comprising therapeutic compound(s) of the disclosure include sterile aqueous or non-aqueous solutions, suspensions, and emulsions. Examples of non-aqueous solvents are propylene glycol, polyethylene glycol, vegetable oils (e.g., olive oil), and injectable organic esters such as ethyl oleate. Examples of aqueous carriers include water, saline, and buffered media, alcoholic/aqueous solutions, and emulsions or suspensions. Examples of parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like. Preservatives and other additives such as, other antimicrobial, anti-oxidants, cheating agents, inert gases and the like also can be included.

Pharmaceutical compositions suitable for injectable use include sterile aqueous solutions (where water soluble) or dispersions and sterile powders for the extemporaneous preparation of sterile injectable solutions or dispersions. In all cases, the composition should be sterile and should be fluid to the extent that easy syringability exists. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be typical to include isotonic agents, for example, sugars, polyalcohols, such as mannitol, sorbitol, or sodium chloride in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

It is especially advantageous to formulate parenteral compositions in dosage unit form for ease of administration and uniformity of dosage. “Dosage unit form” as used herein, refers to physically discrete units suited as unitary dosages for the individual to be treated; each unit containing a predetermined quantity of pharmaceutical composition is calculated to produce the desired therapeutic effect in association with the required pharmaceutical carrier. The specification for the dosage unit forms of the disclosure are related to the characteristics of the pharmaceutical composition and the particular therapeutic effect to be achieve.

The pharmaceutical compositions according to the disclosure may be administered at a therapeutically effective amount either locally or systemically. As used herein, “administering a therapeutically effective amount” is intended to include methods of giving or applying a pharmaceutical composition of the disclosure to a subject that allow the composition to perform its intended therapeutic function. The therapeutically effective amounts will vary according to factors, such as the degree of infection in a subject, the age, sex, and weight of the individual. Dosage regime can be adjusted to provide the optimum therapeutic response. For example, several divided doses can be administered daily or the dose can be proportionally reduced as indicated by the exigencies of the therapeutic situation.

For use in the therapeutic applications described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more therapeutic compounds described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprise therapeutic compounds disclosed herein with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein. These other therapeutic agents may be used, for example, in the amounts indicated in the Physicians' Desk Reference (PDR) or as otherwise determined by one of ordinary skill in the art.

The following examples are intended to illustrate but not limit the disclosure. While they are typical of those that might be used, other procedures known to those skilled in the art may alternatively be used.

### EXAMPLES

Mouse models. All animal procedures were performed in accordance with animal care committee's regulations. Mice were group housed at maximum of five mice per cage under specific pathogen-free conditions with 12 hour (h) light/dark cycle, with free access to food and water. The MeCP2-KO mice (Mecp2 tm1.1Jae) used in this study were created by deleting exon3, containing the methyl-DNA-binding domain of MeCP2 (Chen et al., 2001), and were obtained from Mutant Mouse Resource and Research Centers (MMRRC). MeCP2-KO mice were maintained on a C57Bl/6 background.

Pharmacological treatment of mice. Administration of ADH-503 (MedChemExpress, Cat. #HY-15701B) to MeCP2-KO male mice began at 3 weeks of age. ADH-503 was formulated for treatment in 0.5% carboxymethyl cellulose and 0.18 Tween 80 in sterile water and administered at 120 mg/kg by oral gavage once daily. As per established standards for preclinical studies in MeCP2-KO mice, MeCP2-KO mice were analyzed for lifespan and phenotypic progression. Once a week, progression of the RTT phenotype was monitored using an observational severity scoring. Briefly, mice were scored for mobility, gait, hindlimb clasping, tremor, breathing, and general condition, blind to genotype and treatment status. Each of the six symptoms was given a score of either 0 (absent or the same as wild type), 1 (present) or 2 (severe). These scores were summed up to give a total severity score.

Cell lines. Donated healthy (CTRL) and patients (RTT) fibroblasts were obtained via skin biopsies from patients after informed consent was appropriately given under protocols approved by the University of California, San Diego Institutional Review Board. All experiments were approved and performed following the Institutional Review Boards (IRB) and Embryonic Stem Cell Research Oversight (ESCRO) guidelines and regulations.

Fibroblasts from male RTT (Q83X and N126I) patients and their fathers (WT83 and WT126) were reprogrammed and differentiated into NPCs (Marchetto, et al. Cell, 143:527-39, 2010). Moreover, MeCP2-mutant isogenic cell lines were generated using the clustered regularly-interspaced short palindromic repeats (CRISPR)/CRISPR associated protein 9 (Cas9) genome editing. Isogenic stem cell models of RTT were generated by a frame-shift mutation (also found in an RTT patient) in the MECP2 gene in H1 embryonic stem cells (ESCs). Additionally, a patient cell line was rescued with an early stop codon mutation (Q83X), restoring MeCP2 protein levels to normal using CRISPR/Cas9 genome editing (noted as “KO^{R}”) (Zhang et al., PNAS USA, 113:3185-90, 2016). Cell lines used in different assays are summarized in the table below (Table 1).

All the cell lines tested negative for mycoplasma contamination. All cell lines used have been authenticated. In addition, all cell lines have been genotyped to ensure the presence of the mutation. The absence of the MeCP2 protein in the patient cell line has been verified by immunohistochemistry.

TABLE 1
Summary of cell lines used in each experiment.

| | Cell Lines | | | | | | | | | | | |
|---|---|---|---|---|---|---|---|---|---|---|---|---|
| | | | | | mutant | | CTRL | CTRL | CTRL | | | |
| | CTRL | | Rescue | KO | MECP2 | CTRL | #3 | #3 | #3 | KO | Rescue | CTRL |
| | # 1 | KO# 1 | # 1 | # 2 | #1 | #2 | clone 1 | clone 2 | clone 3 | # 3 | #2 | # 4 |
| Genotype | CTRL | KO | KO rescue | KO | mutant | CTRL | CTRL | CTRL | CTRL | KO | KO rescue | CTRL |
| MGL | ✓ | ✓ | ✓ | ✓ | ✓ | ✓ | ✓ | ✓ | ✓ | ✓ | ✓ | |
| Neuron co-culture | | | | | | | | ✓ | | ✓ | ✓ | |
| Spheroids co- | ✓ | ✓ | | | | ✓ | | ✓ | | ✓ | ✓ | |
| culture | | | | | | | | | | | | |
| Morphometrics | ✓ | ✓ | | | | | ✓ | ✓ | | ✓ | ✓ | |
| Bulk RNA | | | | | | | | ✓ | | ✓ | ✓ | |
| sequencing for | | | | | | | | | | | | |
| MGL | | | | | | | | | | | | |
| Bulk RNA | | | | | | | ✓ | ✓ | | | | ✓ |
| sequencing for | | | | | | | | | | | | |
| neurons | | | | | | | | | | | | |
| Bulk RNA | | | | | | | | ✓ | | | | ✓ |
| sequencing for | | | | | | | | | | | | |
| astrocytes | | | | | | | | | | | | |
| Nano String | | | ✓ | ✓ | | ✓ | ✓ | ✓ | | ✓ | ✓ | |
| ChIP-sequencing | | | | | | | | ✓ | | ✓ | ✓ | |
| Glutamate | ✓ | ✓ | | | | ✓ | ✓ | ✓ | | ✓ | ✓ | |
| Mesoscale | ✓ | ✓ | ✓ | ✓ | | ✓ | ✓ | ✓ | | ✓ | ✓ | |
| ROS | | | | | ✓ | ✓ | | ✓ | | ✓ | ✓ | |
| Chemotaxis | ✓ | ✓ | | | | ✓ | | ✓ | | ✓ | ✓ | |
| Phagocytosis | | | ✓ | ✓ | | | ✓ | ✓ | ✓ | ✓ | ✓ | |
| Phagocytosis | | | | | | | | ✓ | | | ✓ | |
| Compound | | | | | | | | | | | | |
| screening | | | | | | | | | | | | |
| Synaptosome | | | | | | | | ✓ | | | | |
| isolation | | | | | | | | | | | | |
| Proteomics | | | | | | | | ✓ | | ✓ | ✓ | |

Human-induced pluripotent stem cells (hiPSCs), microglia-like cells, NPCs, neurons and cortical spheroids. Human iPSCs cell lines obtained from donors were generated as previously described (Marchetta et al., supra, 2010) by reprogramming fibroblasts. The iPSC colonies were plated on Matrigel-coated (BD Biosciences) plates and maintained in mTESR media (Stem Cell Technologies).

hiPSC-derived microglia-like cells were generated as previously described using a differentiation protocol adapted from Douvaras et al. (Stem Cell Reports, 2017). The iPSC colonies were plated on Matrigel-coated (BD Biosciences) plates and maintained in mTESR media (Stem Cell Technologies). The protocol of myeloid cell lineage consisted of 4 sequential steps. In the first step, primitive streak cells were induced by BMP4 addition, which in step 2, were differentiated into hemangioblast-like hematopoietic precursors [VEGF (80 ng/ml, Peprotech), SCF (100 ng/ml, Gemini) and basic fibroblast growth factor (bFGF), (25 ng/ml, Life Technologies)]. Then, in the third step, the hematopoietic precursors were pushed towards myeloid differentiation [FLT-3 ligand (50 ng/ml, HumanZyme), IL-3 (50 ng/ml, Gemini), SCF (50 ng/ml, Gemini), Thrombopoietin, TPO (5 ng/ml), M-CSF (50 ng/ml)] and finally into the monocytic lineage in step 4 [FLT3-ligand (50 ng/ml), M-CSF (50 ng/ml), GM-CSF (25 ng/ml)]. Microglia-like cell precursors in suspension in step 4 were recovered every 3-4 for 60 days from the conditioned media, sorted by using anti-CD14 magnetic microbeads (MACS, Miltenyi), treated with 50 ng/ml M-CSF and 50 ng/ml IL-34 in M2 media [Neurobasal media supplemented with 2×Gem21 NeuroPlex, 1×NEAA, 1× Glutamax] for a week for microglial differentiation before being used in different experiments. The lipid membranes of CD14⁺ sorted iPSC-derived microglia-like cells were labeled with a red membrane-labeling dye, PKH26 (Sigma-Aldrich), diluted in a 2× solution of Diluent C (Sigma-Aldrich) following the instructions of the manufacturer to assure their presence during the co-culture experiments with neurons or spheroids.

hiPSC-derived NPCs were obtained and maintained as previously described (Marchetto et al, supra, 2010). The iPSCs lines maintained in mTESR media were switched to N2 media (DMEM/F12 media supplemented with 1×N2 NeuroPlex Serum-Free Supplement (Gemini) supplemented with the dual SMAD inhibitors 1 μM of dorsomorphin (Tocris) and 10 μM of SB431542 (Stemgent) daily, for 48 hours. After two days, colonies were scraped off and cultured under agitation (95 rpm) as embryoid bodies (EB) for seven days using N2 media with dorsomorphin and SB431542. Media was changed every other day. EBs were then plated on Matrigel-coated dishes and maintained in DMEM/F12 supplemented with 0.5× of N2 supplement, 0.5×Gem21 NeuroPlex Serum-Free Supplement (Gemini), 20 ng/ml basic fibroblast growth factor (bFGF, LifeTechnologies) and 18 penicillin/streptomycin (P/S). After seven days in culture, rosettes arising from the plated EBs were manually picked, gently dissociated with StemPro Accutase (LifeTechnologies) and plated onto poly-L-ornithine (Sigma)/Laminin-coated (LifeTechnologies) plates. NPCs were maintained in DMEM/F12 with 0.5×N2, 0.5×Gem21, 20 ng/ml bFGF and 1% P/S. The medium was changed every other day. NPCs were split as soon as confluent using StemPro Accutase for 5 min at 37° C., centrifuged and replated with NGF with a 1:3 ratio in poly-L-ornithine/Laminin-coated plates.

hiPSC-derived neurons were obtained from NPCs, differentiated into neurons by adding 10 μM ROCK inhibitor (Y-27632; Calbiochem) in the absence of bFGF for 6-8 weeks. Medium was changed every other day.

Generation of hiPSC-derived cortical spheroids. To generate functional neural networks, growing NPCs were dissociated with Accutase and plated on 6-well dishes (3-5×10⁶ cell per well) under shaker agitation (95 rpm) at 37° C. as previously described (Sharma et al., PNAS, 2019). The media used was DMEM-F12 supplemented with 0.5×GEM21 (Gemini), 0.5×N2 (Gemini), and 20 ng/mL bFGF (Life Technologies). The next day (day 0), neural fate was induced by removing bFGFs and adding 10 μM Rock inhibitor Y-27632 (Tocris). Two days later (day 2), media was changed to fresh media without Rock inhibitor. The differentiation took place for 2 weeks in suspension, with media changed every 4 days. For co-culture experiments, 50,000 microglia-like cells (MGL) were added per spheroids and the MGL were allowed to invade the spheroids for an additional week on the shaker. 3D spheroids with or without MGL were plated on poly-ornithine/Laminin-precoated 12-well multielectrode array (MEA) plates (Axion) with DMEM-F12 media supplemented with 0.5×GEM21, 0.5×N2.

Targeted Gene Expression. Total RNA was extracted from hiPSC-derived microglia-like cells using Qiagen RNeasy Micro Plus kit according to manufacturer's instructions. 50 ng of total RNA was then processed with the NanoString nCounter system (NanoString, Seattle, Washington, USA) per vendor instructions with Human Myeloid Innate Immunity Panel which includes 770 genes related to 19 different pathways to incorporate myeloid cell function and characterization. All samples were normalized to a batch of endogenous housekeeping genes. Data export and normalization were performed using an R-program based nSolver software (NanoString). Data was further analyzed using Rosalind On Ramp software.

Targeted Gene Expression Analysis Methods. Data was analyzed by Rosalind (rosalind.onramp.bio), with a HyperScale architecture developed by OnRamp BioInformatics, Inc. (San Diego, CA). Read Distribution percentages, violin plots, identity heatmaps, and sample MDS plots were generated as part of the QC step. The limma R library was used to calculate fold changes and p-values. Clustering of genes for the final heatmap of differentially expressed genes was done using the PAM (Partitioning Around Medoids) method using the fpc R library that takes into consideration the direction and type of all signals on a pathway, the position, role and type of every gene, etc. Functional enrichment analysis of pathways, gene ontology, domain structure and other ontologies was performed using HOMER. Several database sources were referenced for enrichment analysis, including Interpro, NCBI, KEGG6, MSigDB, REACTOME, WikiPathways. Enrichment was calculated relative to a set of background genes relevant for the experiment. Additional gene enrichment is available from the following partner institutions: Advaita (advaitabio.com/ipathwayguide).

RNA-seq. RNA was isolated from MGL using RNeasy Micro Kit and from hiPSC-derived-neurons and astrocytes using RNeasy Mini Kit. The samples were run on Illumina NovaSeq and HiSeq4000 respectively.

Bulk RNA-seq analysis methods. Data was analyzed by Rosalind (rosalind.onramp.bio), with a HyperScale architecture developed by OnRamp BioInformatics, Inc. (San Diego, CA). Reads were trimmed using cutadapt. Quality scores were assessed using FastQC. Reads were aligned to the Homo sapiens genome build hg19 using STAR. Individual sample reads were quantified using HTseq and normalized via Relative Log Expression (RLE) using DESeq2 R library. Read Distribution percentages, violin plots, identity heatmaps, and sample MDS plots were generated as part of the QC step using RSeQC. DEseq2 was also used to calculate fold changes and p-values and perform optional covariate correction. Clustering of genes for the final heatmap of differentially expressed genes was done using the PAM (Partitioning Around Medoids) method using the fpc R library. Hypergeometric distribution was used to analyze the enrichment of pathways, gene ontology, domain structure, and other ontologies. The topGO R library was used to determine local similarities and dependencies between GO terms in order to perform Elim pruning correction. Several database sources were referenced for enrichment analysis, including Interpro, NCBI, MSigDB, REACTOME, WikiPathways. Enrichment was calculated relative to a set of background genes relevant for the experiment. Functional enrichment analysis of pathways, gene ontology, domain structure and other ontologies was performed using HOMER.

Biological Sample Correlation and Clustering Analysis. Biological replicates subjected to high throughput transcriptome sequencing (RNA-seq) were associated to a reference transcriptome and represented as a matrix of distance having the normalized read counts. The matrix was then used to calculate the Euclidian distance between pairs of samples to calculate and create the Heatmap and the dendrogram. The matrix of distance was also used to create the 3D Principal Component Analysis (PCA) to calculate and represent the maximum variance between samples. The analysis were conducted with the R statistical framework version 3.4.1. Data were normalized with regularized log transformation. Sample correlation and clustering were performed with Plotly for R package.

Liquid Chromatography-Mass Spectrometry (LC-MS) label-free Proteomic Analysis. Sample preparation: To prepare MGL for proteomics, cells were pelleted and flash-frozen.

Protein samples were diluted in TNE (50 mM Tris pH 8.0, 100 mM NaCl, 1 mM EDTA) buffer. RapiGest SF reagent (Waters Corp.) was added to the mix to a final concentration of 0.18 and samples were boiled for 5 min. TCEP (Tris (2-carboxyethyl) phosphine) was added to 1 mM (final concentration) and the samples were incubated at 37° C. for 30 min. Subsequently, the samples were carboxymethylated with 0.5 mg/ml of iodoacetamide for 30 min at 37° C. followed by neutralization with 2 mM TCEP (final concentration). Proteins samples prepared as above were digested with trypsin (trypsin:protein ratio—1:50) overnight at 37° C. RapiGest was degraded and removed by treating the samples with 250 mM HCl at 37° C. for 1 h followed by centrifugation at 14000 rpm for 30 min at 4° C. The soluble fraction was then added to a new tube and the peptides were extracted and desalted using C18 desalting columns (Thermo Scientific, PI-87782). Peptides were quantified using BCA assay and a total of 1 μg of peptides were injected for LC-MS analysis.

LC-MS-MS: Trypsin-digested peptides were analyzed by ultra high-pressure liquid chromatography (UPLC) coupled with tandem mass spectroscopy (LC-MS/MS) using nano-spray ionization. The nanospray ionization experiments were performed using a Orbitrap fusion Lumos hybrid mass spectrometer (Thermo) interfaced with nano-scale reversed-phase UPLC (Thermo Dionex UltiMate™ 3000 RSLC nano System) using a 25 cm, 75-micron ID glass capillary packed with 1.7-μm C18 (130) BEH™ beads (Waters corporation). Peptides were eluted from the C18 column into the mass spectrometer using a linear gradient (5-80%) of ACN (Acetonitrile) at a flow rate of 375 l/min for 2 h. The buffers used to create the ACN gradient were: Buffer A (98% H2O, 2% ACN, 0.18 formic acid) and Buffer B (100% ACN, 0.1% formic acid). Mass spectrometer parameters are as follows; an MS1 survey scan using the orbitrap detector (mass range (m/z): 400-1500 (using quadrupole isolation), 120000 resolution setting, spray voltage of 2200 V, Ion transfer tube temperature of 275 C, AGC target of 400000, and maximum injection time of 50 ms) was followed by data dependent scans (top speed for most intense ions, with charge state set to only include +2-5 ions, and 5 second exclusion time, while selecting ions with minimal intensities of 50,000 at in which the collision event was carried out in the high energy collision cell (HCD Collision Energy of 30%), and the fragment masses where analyzed in the ion trap mass analyzer (With ion trap scan rate of turbo, first mass m/z was 100, AGC Target 5000 and maximum injection time of 35 ms).

Data analysis: Protein identification and label free quantification was carried out using Peaks Studio 8.5 (Bioinformatics solutions Inc.). The software was configured to work with PTM (Post-translational modification) data analysis. For protein identification, it is generated the PEAKS peptide score (−10 lgP), which is calculated for every peptide-spectrum match (PSM). The score is derived from the p-value that indicates the statistical significance of the peptide-spectrum match. PSM values are then used for statistical analysis, that was based on two-tail p-value and is calculated as the significance of the feature vector using the log-normal distribution corresponding to its quality and its maximum group ratio.

### Ingenuity Pathway Analysis.

The functional network analyses were generated through the use of IPA (QIAGEN Inc., qiagenbio-informatics.com/products/ingenuity-pathway-analysis).

Morphometric Analysis of microglia-like cells. To analyze the morphology of microglia-like cells, 20,000 cells were plated and allowed to differentiate for 7 days. Three pictures per well and at least three wells per genotype were taken for the analysis. Results were analyzed by the Animantis, LLC group (San Diego, CA). Briefly, visible surface area of the cell was reported in μm² (calculated based on the scale bar in the raw images). For the eccentricity, a value capturing how much a cell's shape deviates from ‘round’. It is calculated using the ‘major-axis’ and ‘minor-axis’ of the detected cell; the major-axis is the widest part of the cell, and the minor axis is the width perpendicular to the major axis. Based on these measurements, the formula for eccentricity is:

$$eccentricity = \frac{\sqrt{{major}^{2} - {minor}^{2}}}{major}$$

Note that this formula implies that a circular cell (major and minor axes are the same) would have eccentricity=0, while eccentricity=1 would imply an impossibly flat cell that has minor-axis=0. This is a label automatically applied to the cell that can be one of three options: “round”, a cell that is not overly large and is roughly round; “elongated”, a cell that is not overly large and is not round and finally “clumps”, a cellular object that has been detected but is too large to likely be a single cell.

ChIP-Seq Assay. Chromatin Immunoprecipitation: CTRL, KO and KO^{R} MGL from two different batched were pooled and fixed with 1% formaldehyde for 15 min and quenched with 0.125 M glycine. Chromatin was isolated by the addition of lysis buffer, followed by disruption with a Dounce homogenizer. Lysates were sonicated and the DNA sheared to an average length of 300-500 bp. Genomic DNA (Input) was prepared by treating aliquots of chromatin with RNase, proteinase K and heat for de-crosslinking, followed by ethanol precipitation. Pellets were resuspended and the resulting DNA was quantified on a NanoDrop spectrophotometer. Extrapolation to the original chromatin volume allowed quantitation of the total chromatin yield.

An aliquot of chromatin (30 μg) was precleared with protein A agarose beads (Invitrogen). Genomic DNA regions of interest were isolated using 4 μg of antibody against MeCP2 (Diagenode, cat #C15410052, lot #A20-0042). Complexes were washed, eluted from the beads with SDS buffer, and subjected to RNase and proteinase K treatment. Crosslinks were reversed by incubation overnight at 65° C., and ChIP DNA was purified by phenol-chloroform extraction and ethanol precipitation.

ChIP Sequencing (Illumina): Illumina sequencing libraries were prepared from the ChIP and Input DNAs by the standard consecutive enzymatic steps of end-polishing, dA-addition, and adaptor ligation. Steps were performed on an automated system (Apollo 342, Wafergen Biosystems/Takara). After a final PCR amplification step, the resulting DNA libraries were quantified and sequenced on Illumina's NextSeq 500 (75 nt reads, single end). Reads were aligned to the human genome (hg38) using the BWA algorithm (default settings). Duplicate reads were removed and only uniquely mapped reads (mapping quality >=25) were used for further analysis. Alignments were extended in silico at their 3′-ends to a length of 200 bp, which is the average genomic fragment length in the size-selected library, and assigned to 32-nt bins along the genome. The resulting histograms (genomic “signal maps”) were stored in bigWig files. Peak locations were determined using the MACS algorithm (v2.1.0) with a cutoff of p-value=1e-7. Peaks that were on the ENCODE blacklist of known false ChIP-Seq peaks were removed. Signal maps and peak locations were used as input data to Active Motifs proprietary analysis program, which creates Excel tables containing detailed information on sample comparison, peak metrics, peak locations and gene annotations. KO samples did not generate any sequencing libraries.

ChIP-seq Methods. Data was analyzed by Rosalind (rosalind.onramp.bio), with a HyperScale architecture developed by OnRamp BioInformatics, Inc. (San Diego, CA). Reads were trimmed using cutadapt. Quality scores were assessed using FastQC. Reads were aligned to the Homo sapiens genome build hg19 using bowtie2. Per-sample quality assessment plots were generated with HOMER and Mosaics. Peaks were called using MACS2 (with input/IgG controls background subtracted, if provided). Peak overlaps and differential binding were calculated using the DiffBind R library. Differential binding was calculated at gene promoter sites. Read distribution percentages, identity heatmaps, and FRiP plots were generated as part of the QC step using ChIPQC R library and HOMER. HOMER was also used to generate known and de novo motifs and perform functional enrichment analysis of pathways, gene ontology, domain structure and other ontologies.

Cell Viability. The cell viability was measured after staining with Trypan Blue (Life technologies) using an automated BioRad cell counter. Pre-MGL were collected from the supernatant and the viability every 3-4 days during the time of the differentiation.

Multielectrode Array Recordings (MEA). Spontaneous action potential activity was recorded by using Axion Biosystems.

BCO and spheroids co-cultured with or without microglia-like cells that had been plated on the multielectrode arrays (MEAs). BCO activity was recorded 2 months after adding MGL and plating onto MEA plates. Spheroid activity was recorded 8 days after plating. MEA channels with similar number and density of cells, and with more than 10 spikes in a 5-min interval were included in the analysis. Recordings were analyzed with the MATLAB-based Neural Metric Tool Software Axis (Axion Biosystems).

Phagocytosis Assay. Phagocytosis assay was performed according to the manufacturer's instructions with pHrodo™ Red Zymosan A BioParticles® (Life Technologies, Carlsbad, USA) conjugate for phagocytosis. Briefly, 20,000 MGL were plated on a 96-well tissue culture plate and incubated in M2 media [Neurobasal media supplemented with 2×Gem21 NeuroPlex, 1×NEAA, 1× Glutamax] containing fluorescently labeled zymosan particles (0.125 mg/ml) for 2 h at 37° C., 5% CO₂. Fluorescence was measured with Tecan Infinite 200 PRO microplate reader (Life Sciences, Switzerland).

For experiments featuring block of zymosan uptake, cell monolayers were treated with 1 μM Cytochalasin D (Sigma-Aldrich) 30 min prior to incubation with zymosan.

For the compound screening, MGL were pre-treated with each compound one day prior to the phagocytosis assay. All compounds were used at 10 μM, which did not cause any decrease in cell viability.

Brain Cortical Organoid-derived synaptosome-enriched fraction isolation. Synaptosomes were isolated from 6 weeks-old brain organoids from CTRL samples generated from our previously published protocol (using Syn-PER™ Synaptic Protein Extraction Reagent (Thermo Fisher) according to the manufacturer's instructions. Synaptosomes were then conjugated using IncuCyte® pHrodo® Red Cell Labeling Kit for Phagocytosis and added on top of MGL for 2 hours. The fluorescence was measured using the Incucyte software.

Glutamate Assay. The Glutamate Colorimetric Assay kit (ab83389, Abcam, USA) was used to measure the glutamate in the conditioned media by microglia-like cells following the manufacturer's instructions. 50,000 microglia-like cells were plated in 100 μl of media and conditioned for 24 hours. 50 μl of samples was used.

Reactive Oxygen Species (ROS) Assay. 50,000 microglia-like cells were plated in 100 μl of media and conditioned for 24 hours. ROS-Glo H₂O₂ Assay (Promega, USA) was used to measure the reactive oxygen species in the conditioned media by microglia-like cells according to the manufacturer's instructions. The relative luminescence unit (RLU) was recorded in a plate reader.

Image analysis. ImageJ software was used to calculate the integrated density of MeCP2 (antibody information is provided below in immunocytochemistry section) in MGL. Briefly, the channels were split and the integrated density of the appropriate channel was measured by the software.

Chemotaxis Assay. Chemotaxis assay was performed using 5 μm polyester transwell chamber, with 300 mM ATP or CX3CL1 at 100 ng/ml for four hours. The cells that have migrated through the chamber were then manually counted in several regions of interest (ROI).

Immunocytochemistry. Mice were anesthetized and perfused trans-cardiac with PBS followed by 48 buffered paraformaldehyde (PFA) at 8 weeks. Brains were removed and fixed with 48 PFA overnight at 4° C. The fixed tissue was cryoprotected with 30% sucrose and frozen in optimal cutting temperature medium (OCT). Coronal sections were obtained by sectioning the tissue at 30 μm using a microtome. The images were taken in the cerebral cortex region.

Microglia-like cells (MGL), neuron-MGL, neuron-fibroblast and neurons with MGL conditioned media were fixed with 48 paraformaldehyde for 20 min at room temperature. Cells were permeabilized and incubated with blocking solution [10% fetal bovine serum (Life Technologies), 0.1% (v/v) Triton X-100 in 1× Dulbecco's Phosphate Buffer Solution (DPBS, Gibco)] for 30 minutes. Then, the primary antibody was added (diluted in blocking solution) and samples were incubated overnight at 4° C. Cells were then washed two times with 1×-PBS, and incubated with the secondary antibody for one hour at room temperature. Secondary antibodies (all conjugated to Alexa Fluor 488, 555 and 647) were purchased from Life Technologies and used at a 1:1000 dilution. Cells were washed twice (1×-PBS), incubated for 5 min with and fluorescent nuclear 6-diamidino-2-phenylindole (DAPI, VWR International, 1:5,000) for 10 minutes DAPI stain, and mounted with Prolong gold anti-fade reagent (Life Technologies). Samples were imaged using an Axio Observer Z1 Microscope with ApoTome (Zeiss).

Antibodies and dilutions used: Monoclonal Mouse Anti-Human CD68, (1:500, Dako); Polyclonal Rabbit Anti-Iba-1, (1:500, Wako), Polyclonal Rabbit Anti-Human PU.1 (1:500; Cell Signaling Technology), Polyclonal Rabbit Anti-CX3CR1 (1:2000, Biorad), Polyclonal Goat Anti-Human TREM2 (1:100; R&D Systems), Monoclonal Mouse Anti-Human CD11b (1:500, BD Biosciences), Polyclonal Rabbit Anti-Human P2YR12 (1:125, Alomone), Polyclonal Rabbit Anti-Human MeCP2 (1:500 Diagenode), anti-Homer1 (Synaptic Systems, 1:500), anti-VGLUT1 (Synaptic Systems, 1:500), anti-MAP2 (Abcam, 1:2000) and NeuN (Millipore, 1:500).

Co-culture experiments. For neuron-MGL or primary control fibroblast co-culture experiments, 50,000 NPCs were plated on PO-Laminin coated coverslips in NPC media. The next day, the bFGF and added Rock inhibitor were retrieved to start neuronal differentiation. 50,000 MGL progenitor cells freshly sorted with CD14⁺ microbeads or 50,000 primary fibroblasts were directly added onto each coverslip. The neurons were allowed to differentiate for 6-8 weeks in the presence of MGL or fibroblast before proceeding with the synaptogenesis assays. The fibroblasts and the MGL were first labeled with the membrane dye PKH26 to verify their presence at the end of the 8 weeks of neuronal differentiation. Then, another batch of co-culture was generated, this time without the membrane dye, to be able to perform the immunostainings for synaptic proteins without interference from the fluorescent dye. For neurons with MGL conditioned media, 50,000 MGL were plated per 96-well plate and conditioned for 48 hours in neuronal media supplemented with 50 ng/ml IL34 and 50 ng/ml M-CSF. Conditioned media from each well was recovered, filtered using 40 μm cell strainer (BD Biosciences). 100 μl of filtered MGL conditioned media was added onto each coverslip with neurons every two days for 6-8 weeks.

Synapse Formation Assay. hiPSC-derived neurons co-cultured with MGL, fibroblasts or with MGL conditioned media were fixed at 6-8 weeks after bFGF retrieval and imaged using an Axio Observer Z1 Microscope with Apotome (Zeiss) using compiled z-stack images at an objective resolution of 63X. Co-localization of pre-(VGLUT1) and post-synaptic (HOMER1) markers were quantified manually when in contact with MAP2 at a length of 50 μm. Each co-culture combination had at least 10 to 15 images processed from which 10 to 20 MAP2-positive neurons were used for co-localized synaptic puncta quantification from a total of four biological replicated and two independent co-cultured batches.

Neurite tracing. Images used for neurite tracing were taken from an Axio Observer Z1 Microscope with Apotome (Zeiss) using compiled z-stack images at an objective resolution of 63×. iPSC-derived neurons were immunostained using microtubule-associated protein 2 (MAP2) as a neuronal marker to indicate neurite outgrowth. MAP2-positive neurites were then manually traced using the software, ImageJ, with the plugin extension, NeuronJ. Manual traces began at the starting point of the MAP2-positive neurite and were followed along the path until where the neurite terminates. Branches were traced in the same way. Distance was calibrated at a scale of 9.757 pixels/μm.

Mesoscale Assay. CD14⁺ sorted iPSC-derived microglia-like cells were seeded at 5×10⁴ cells per well on 96-well cell culture plates and kept in M2 media. Cells were stimulated with or without treatments of 1 μg/mL E. coli Lipopolysaccharide (LPS, Sigma-Aldrich) for 30 hours. 100 uL of conditioned media was recovered from each well the next day. Cytokine levels were quantified using a customized V-PLEX Human Cytokine 30-Plex Kit (Meso Scale Discovery, Rockville, Maryland, USA) according to the manufacturer's instructions. Chemokines and cytokines measured from custom multiplex panels consist of: eotaxin, eotaxin-3, IL8 (HA), IP-10, MCP-1, MDC, MIP-1α, MIP-1β, TARC, GM-CSF, IL-12/IL-23p40, IL-15, IL-16, IL-17A, IL-1α, IL-5, IL-7, TNFβ, VEGF-A, IL-10, IL-12p70, IL-13, IL-1β, IL-2, TNFα, IL-6. Samples with or without LPS stimulation underwent a 10-fold or 2-fold dilution, respectively.

Statistical Analyses. Results were analyzed using Prism Software (version 6, GraphPad, USA). Statistical significance was determined using one-way ANOVA tests followed by Tukey or Sidak multiple comparisons tests to compare different groups with one variable, or two-way ANOVA tests when there were two variables and Student's t-test to compare means of two groups using a p<0.05. The reported values are means ±SEM, as mentioned in relevant figure captions. Sample sizes, n, reported in relevant figures (as data points) or figure legends.

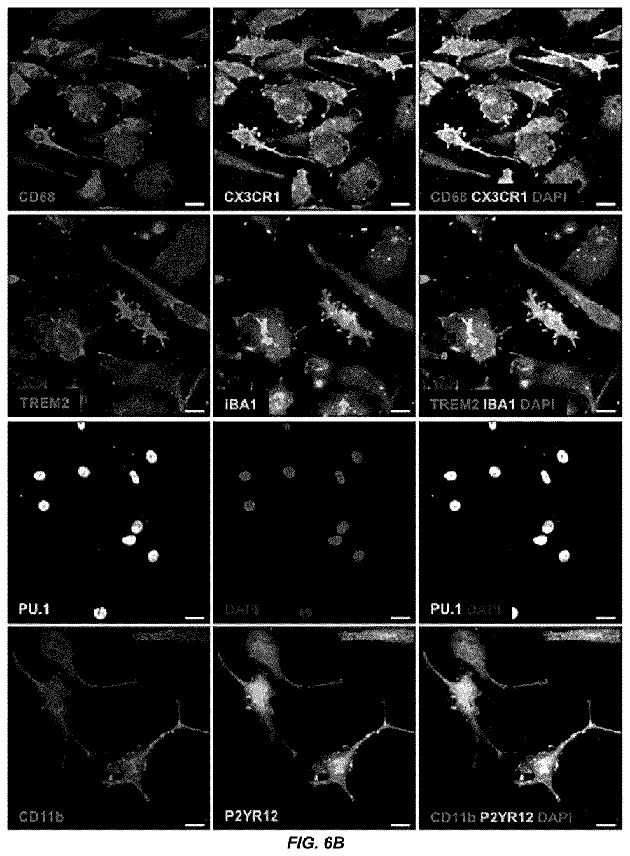

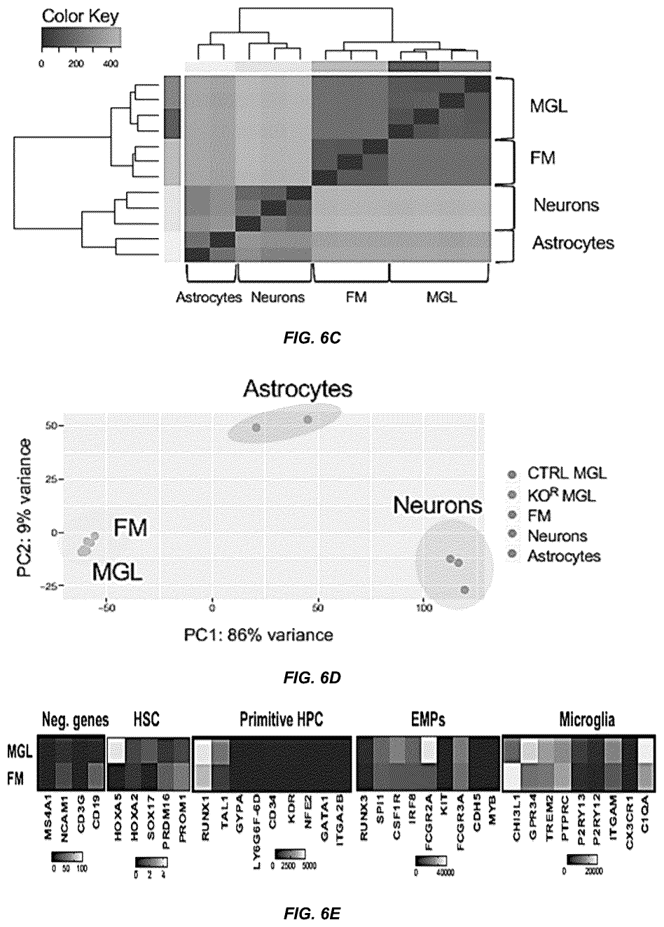

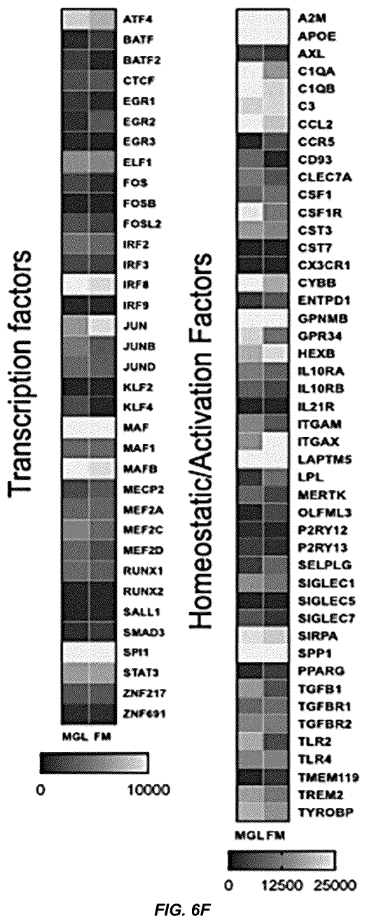

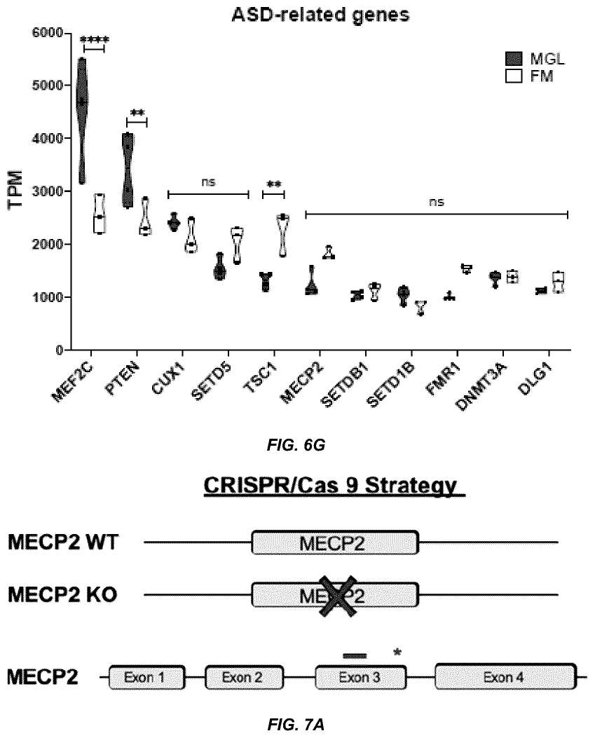

Characterization of human iPSC-derived MGL. Efficient protocols to generate microglia-like cells (MGL) from iPSCs are depicted in FIG. 6A. The protocol makes precursors cells of MGL (pre-MGL) in suspension that are further differentiated into MGL cells after CD14 sorting and treatment with IL-34 and M-CSF for a week (FIG. 6A), generating a homogeneous population of MGL expressing classical microglial markers; CD68, CX3CR1, TREM2, IBA1, PU. 1, CD11b and P2YR12 (FIG. 6B). The MGLs closely recapitulated the transcriptomic signature of human primary fetal microglia (FM), both at the level of microglial transcriptional factors and also homeostatic/activation factors (FIG. 6C). MGL clustered closely with FM compared to iPSC-derived neurons and astrocytes (FIG. 6D-E). MGL highly expressed microglial genes, and at a lesser but similar fashion to FM, genes typically expressed by hematopoietic stem cells (HSC), primitive hematopoietic progenitor cells (HPC), erythromyeloid precursors (EMPs), and did not express the negatively associated genes such as MS4A1, NCAM1, CD3G and CD19 (FIG. 6F). As hypothesized microglial cells can play a role in neurodevelopment, as compared with expression of several ASD-related genes between MGL and FM that revealed similar expression levels (FIG. 6G).

The absence of MECP2 in MGL leads to decreased cell viability, morphological and transcriptional changes. Given that MGL and FM revealed similar expression levels of ASD-related genes, experiments were conducted to determine how MGL could affect neurodevelopment. To address this question, a model of neuronal development perturbation was used by taking advantage of the similarities observed between FM and MGL regarding the expression of several ASD-related genes. The Methyl-CpG Binding Protein 2 (MECP2) is an undoubtfully important gene for neural development, implicated in ASD and several other human conditions. Still, the impact of MECP2 mutations in human microglia during development remains unknown.

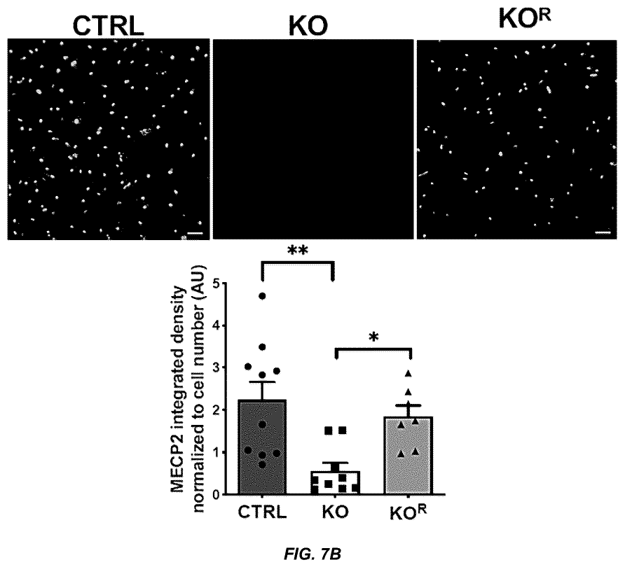

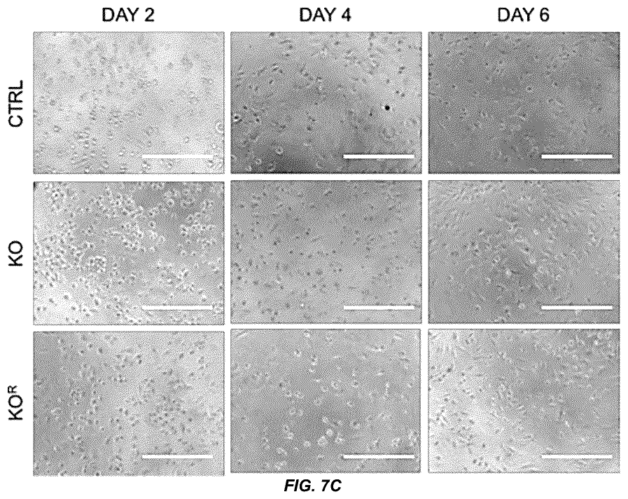

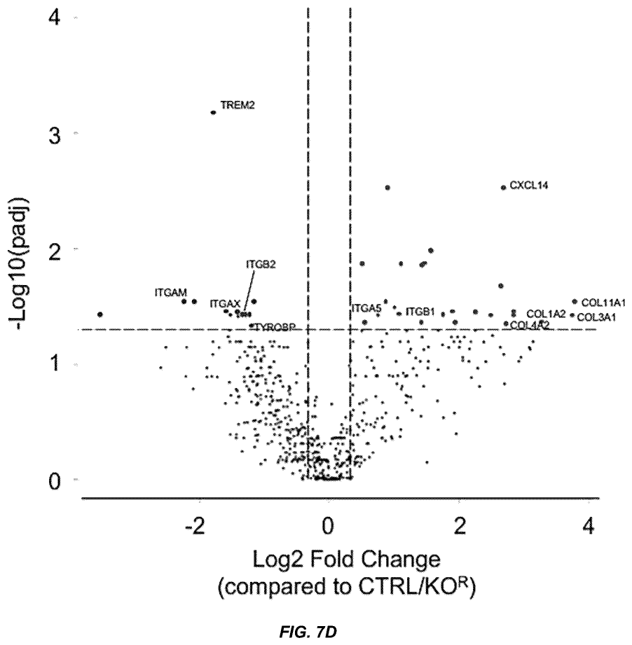

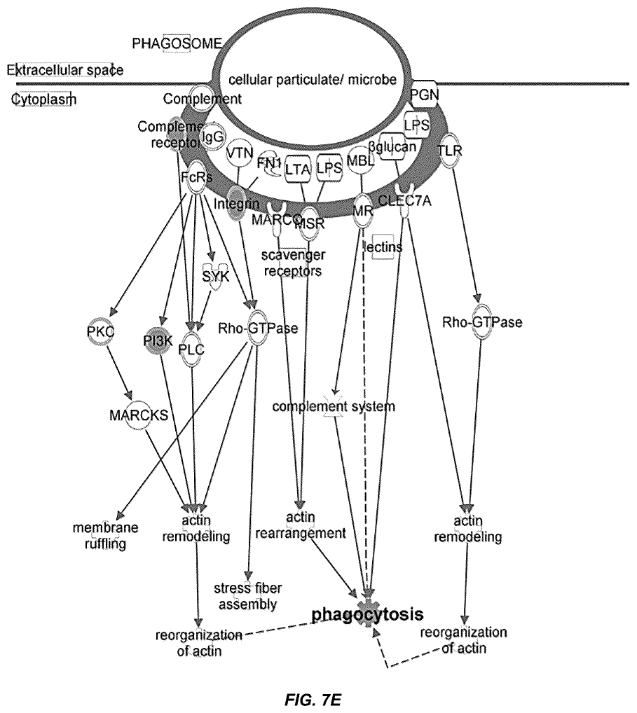

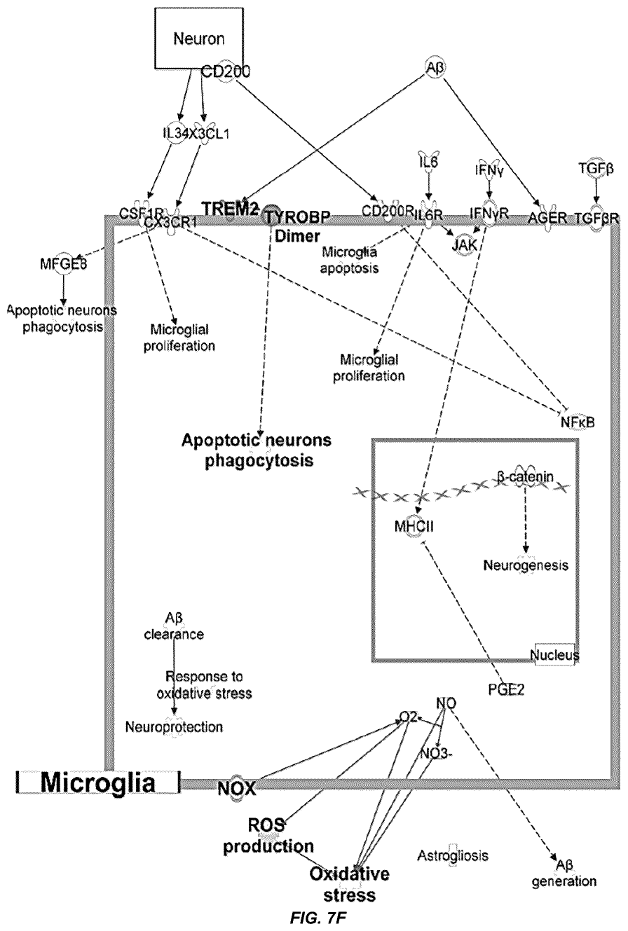

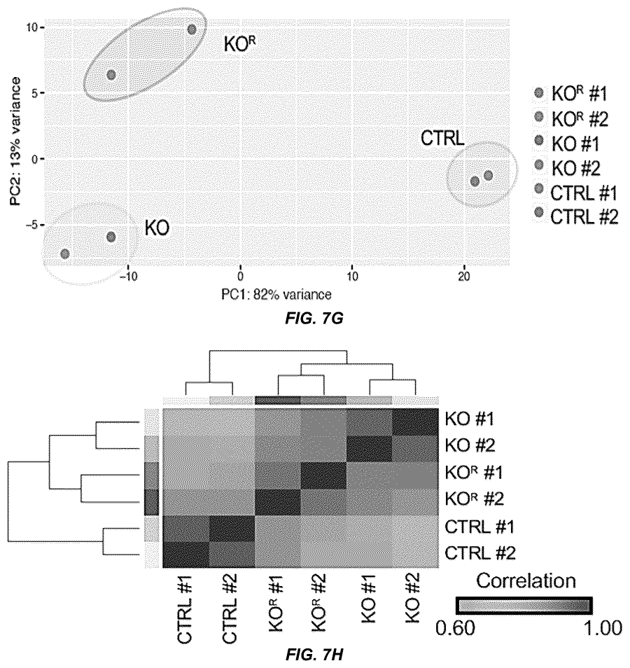

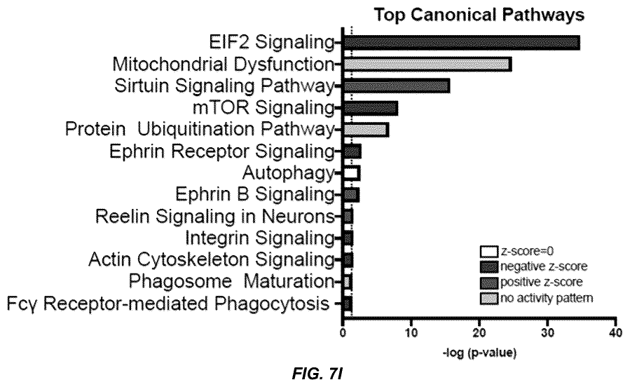

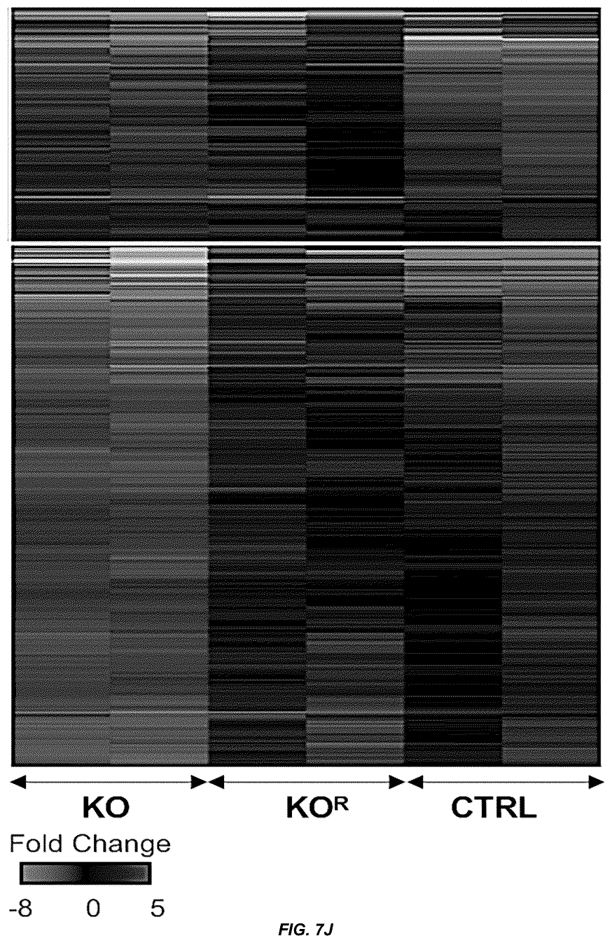

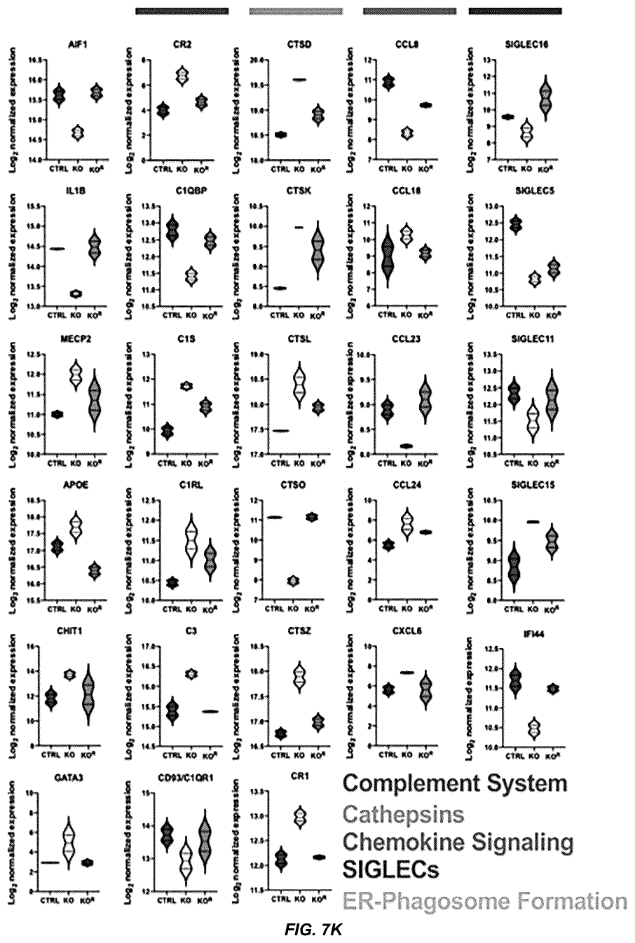

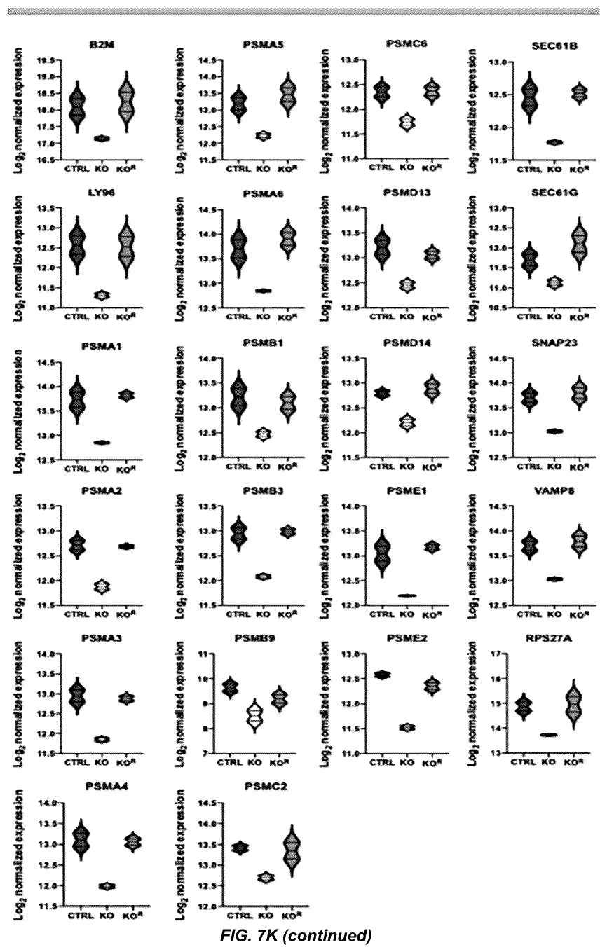

A patient-derived iPSC lines was selected that lacked the MECP2 protein, denoted as “KO,” and generated isogenic CRISPR-corrected rescue lines by restoring the mutation, noted as “KO^{R}” (FIG. 7A). Cell lines obtained from healthy donors (controls), indicated as “CTRL” (see Table 1) were also used.

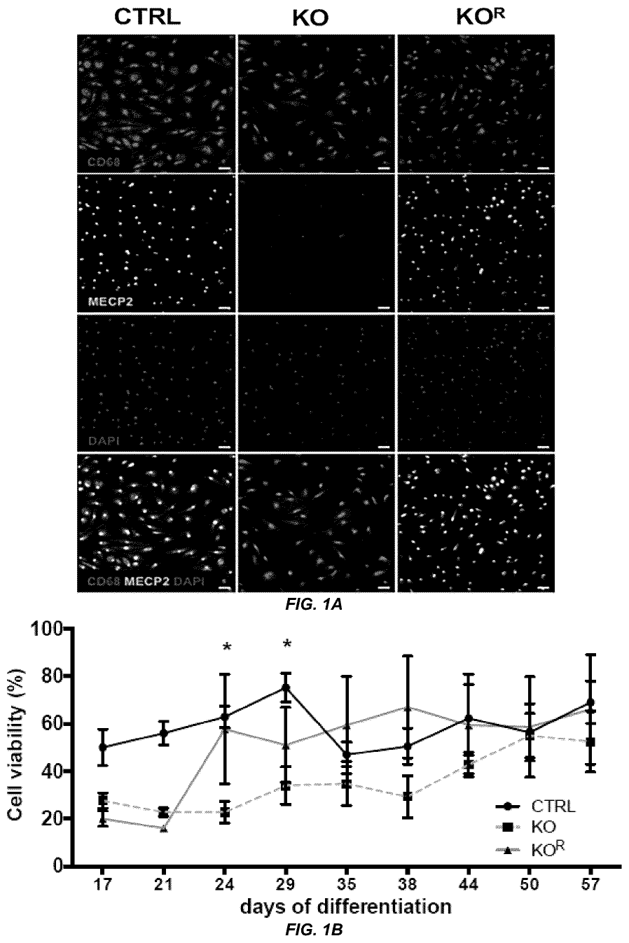

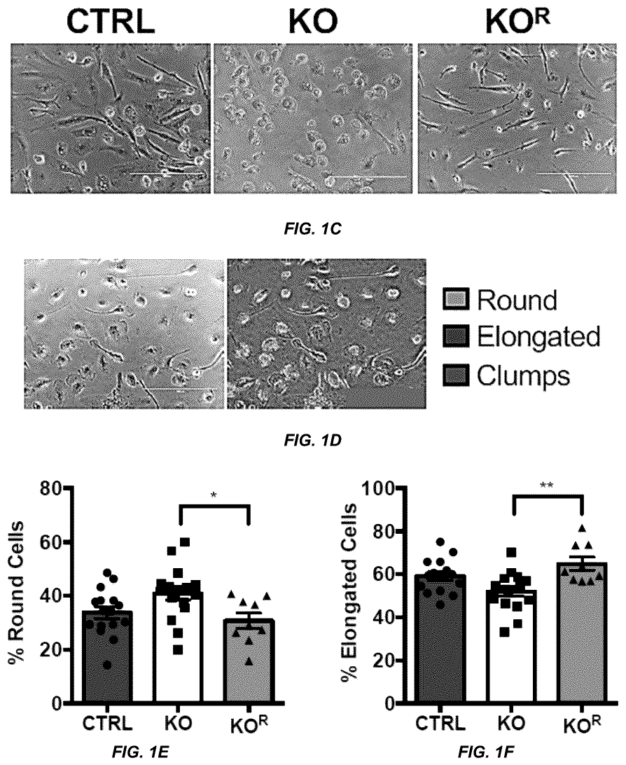

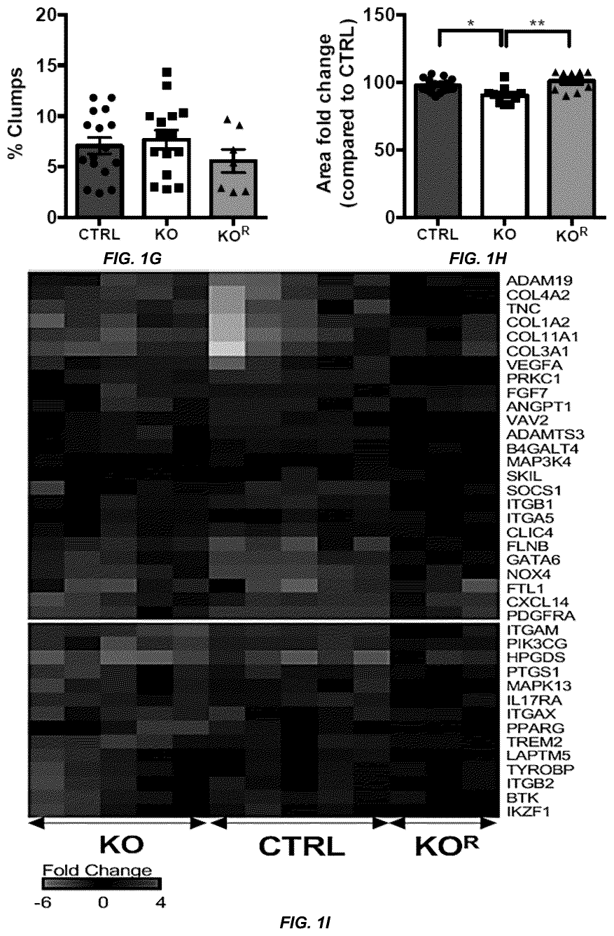

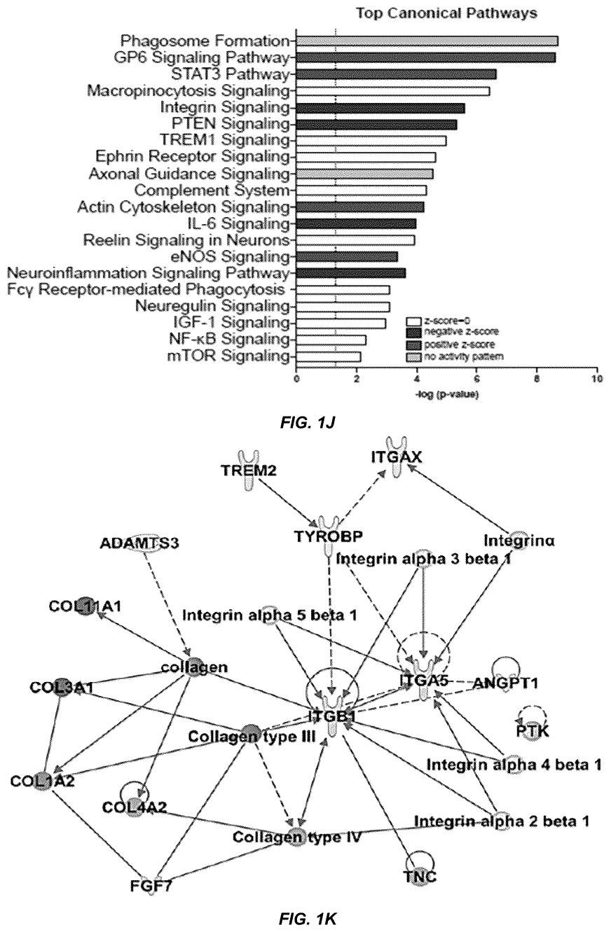

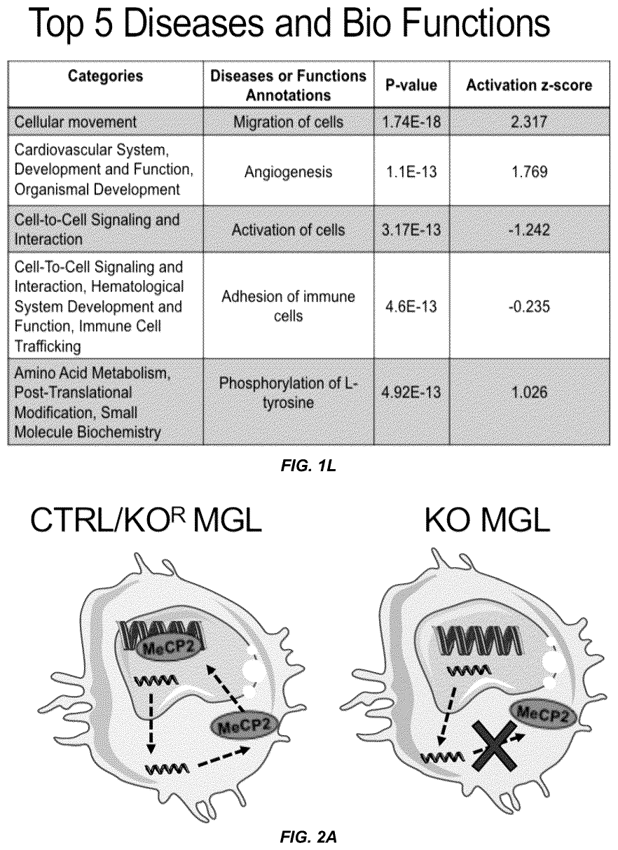

Immunofluorescence analysis confirmed that MGL expressed MECP2, as well as loss of the protein in MGL KO lines and its re-expression on the KO^{R} MGL (FIGS. 1A and 7B). During MGL differentiation, a period of significant latency in the generation of viable KO pre-MGL was observed, occurring from day 24 to day 29 (FIG. 1B), which later caught up with the CTRL and KO^{R} MGL. This effect was linked to a lack of functional MECP2 since KO^{R} lines did not differ from CTRL MGL in the generation of MGL (FIG. 1B). Pre-MGL generated from CTRL, KO, and KO^{R} lines isolated from the media in suspension were then further differentiated into MGL (FIGS. 1C and 7C), and their morphology classified into three groups: round, elongated, or cell clumps (FIG. 1D). In the absence of any external stimuli, both CTRL and KO^{R} MGL generated approximately 60% elongated shaped cells, 35% round cells and around 5% clumped cells (FIG. 1E-G), while KO MGL generated about 50% of elongated shaped and 40% of round-shaped cells (FIG. 1E-G), suggesting the absence of MECP2 decreases the degree of ramification. Also, KO MGL exhibited a smaller surface area compared to CTRL or KO^{R} MGL (FIG. 1H). Alterations in morphology were attributable to the absence of MeCP2, as KO^{R} and CTRL MGL surface areas were indistinguishable (FIG. 1E-H).

To address whether the differences observed in KO MGL regarding microglial morphology could potentially reflect functional differences, a targeted multiplex transcriptomic analysis was performed using Human Myeloid Innate Immunity Panel gene expression array (NanoString Technologies), which includes 770 genes from 19 different pathways and processes (FIGS. 1I and 7D). KO^{R} lines re-expressing the MECP2 protein, clustered tightly with CTRL MGL lines, confirming observed gene expression changes were linked to loss of MECP2 (FIG. 1A, I). Compared to CTRL and KO^{R} MGL lines, 39 genes were differentially expressed in KO MGL lines with a p-value <0.05 and fold-change greater than 1.25, (FIGS. 1I and 7D). Using Ingenuity Pathway Analysis (IPA), the top canonical pathways related to the CNS were identified as phagosome formation, integrin signaling, Fcγ receptor-mediated phagocytosis in macrophages and monocytes (FIG. 7E), neuroinflammation (FIG. 7F), axonal guidance, TREM1 signaling, complement system and actin cytoskeleton signaling pathways (FIG. 1J). The molecular network obtained through IPA analyses showed reciprocal interactions between the majority of dysregulated genes in KO MGL, suggesting converging molecular pathways (FIG. 1J-L), including those involving cell movement, cell-to-cell signaling and cell adhesion (FIG. 1L).

To detect additional pathway alterations caused by MECP2 loss beyond targeted myeloid genes and function transcriptomics, an unbiased high-throughput messenger RNA sequencing (RNA-seq) was performed on CTRL, KO^{R}, and KO MGL (FIG. 7G-K). These global transcriptome analyses revealed 2513 differentially expressed genes (DEG) with a p-value <0.05 and a fold-change greater than 1.25 (FIG. 7J). Four genes were identified in common between the two transcriptomic analyses; COL1A2, ITGAX, PTGS1, and TNC; two of which, COL1A2 and TNC were both upregulated in targeted and untargeted transcriptome analyses; as well as shared canonical pathways, such as integrin signaling, actin cytoskeleton signaling, phagosome formation and Fcγ receptor-mediated phagocytosis in macrophages and monocytes (FIG. 1I-J, FIG. 7I). Several other gene families belonging to the complement system, cathepsins, chemokine signaling, SIGLECs, and ER-phagosome formation were also found to be dysregulated between KO and CTRL/KO^{R} MGL (FIG. 7K). Collectively, the transcriptomic analyses, using both targeted and high throughput methods, provided converging evidence that loss of MECP2 alters phagocytosis pathways.

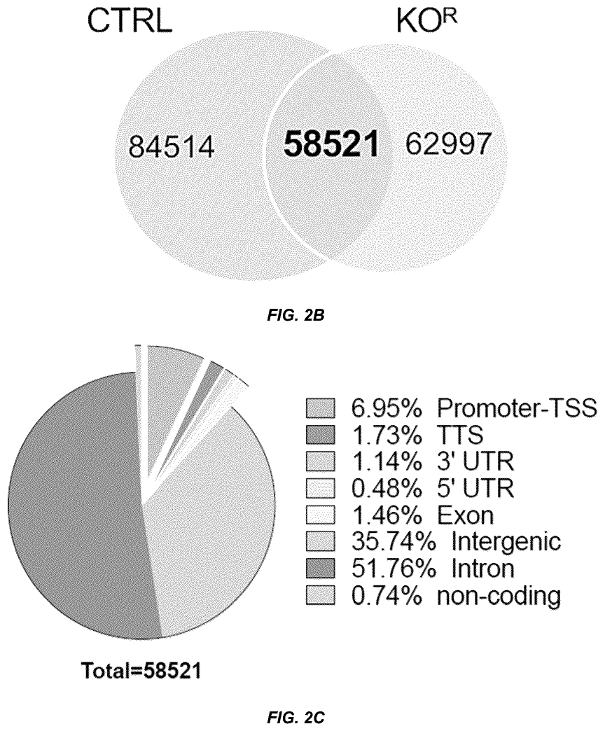

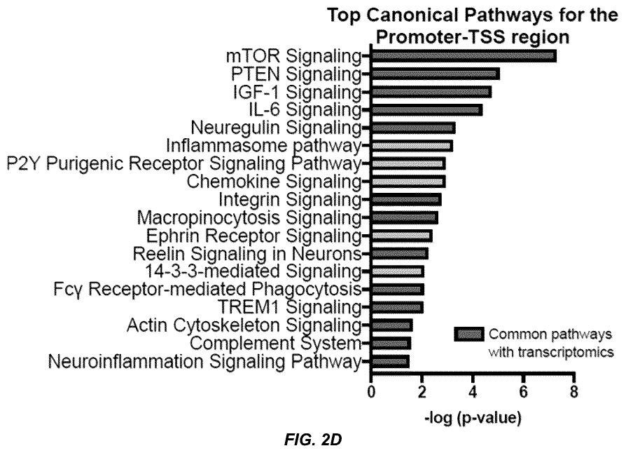

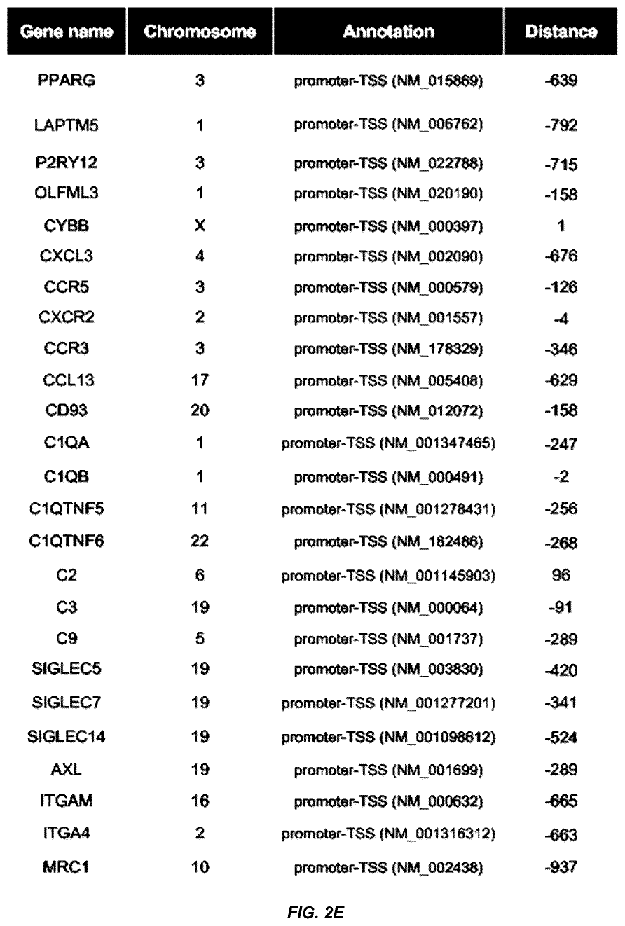

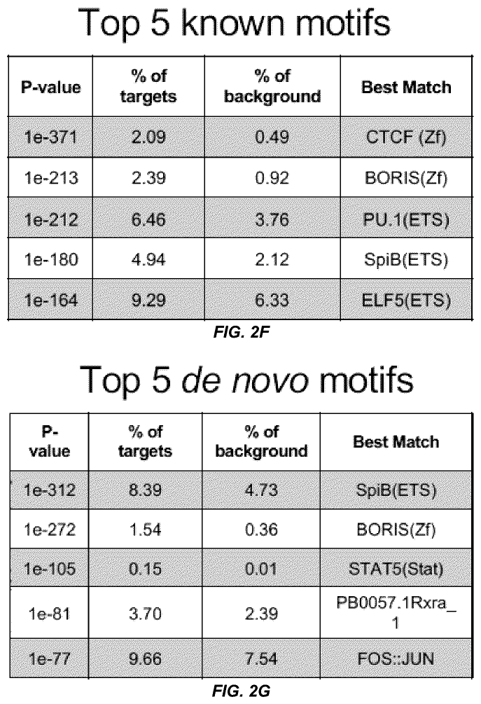

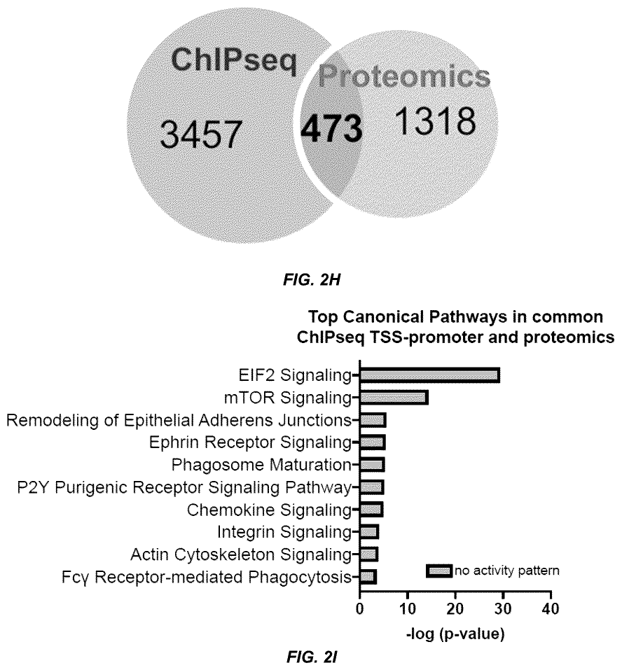

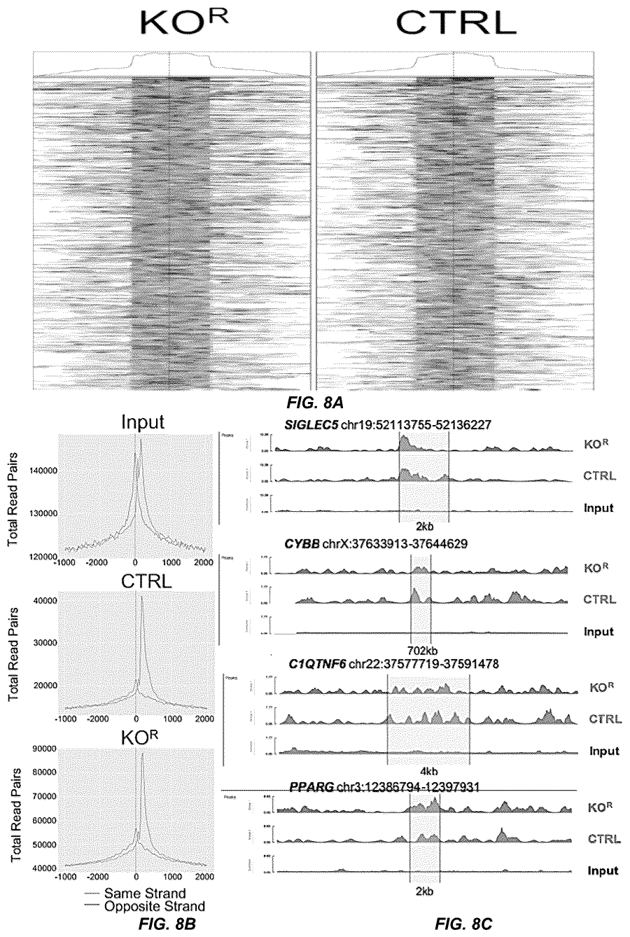

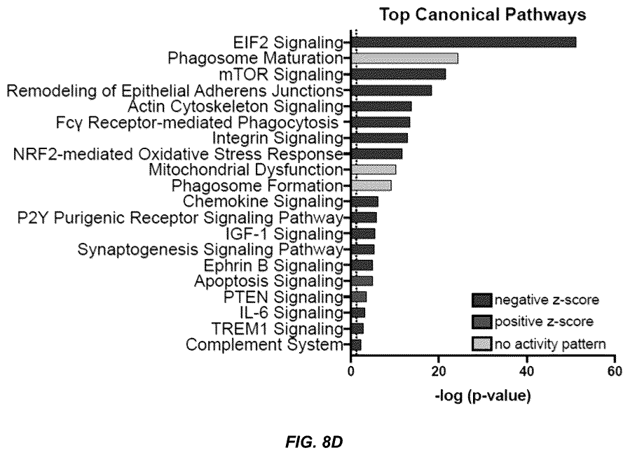

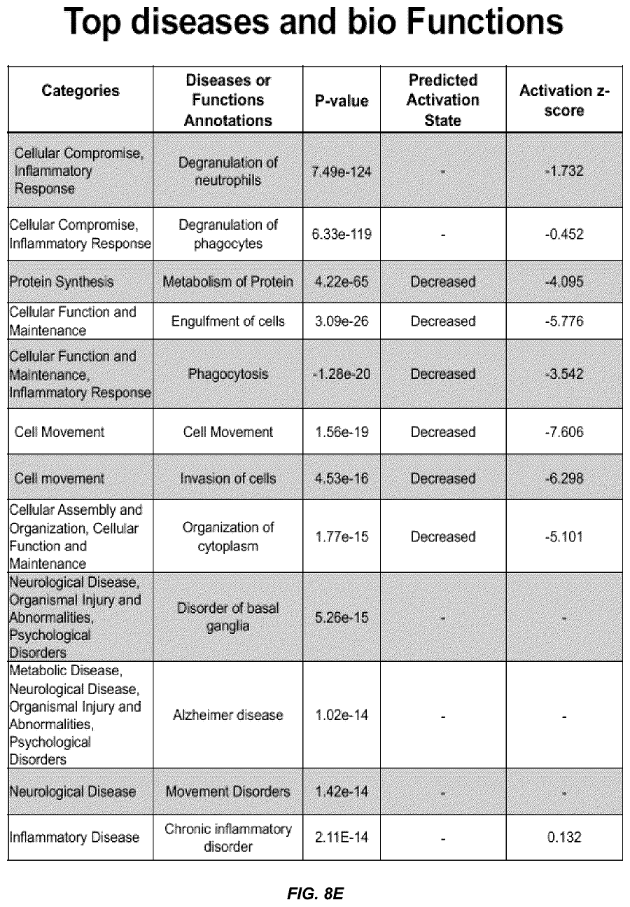

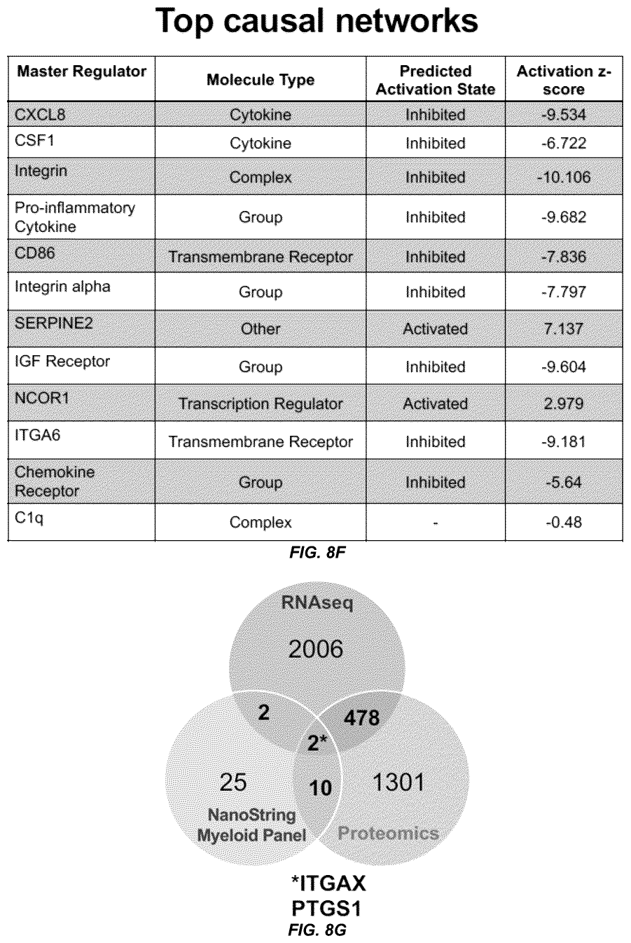

MECP2 Chromatin-immunoprecipitation sequencing and proteomic assays on MGL. To assess whether MECP2 could directly regulate microglial function by occupying target genes or their promoters, a MECP2 chromatin immuno-precipitation sequencing assay (MECP2 ChIP-seq) was performed on KO^{R} and CTRL MGL (FIG. 2A-G, FIG. 8A-C). As a negative control, KO MGL were used and were unable to generate sequencing libraries due to the absence of MeCP2 protein and/or binding. Over 120,000 MECP2 occupation sites were identified genome-wide on both KO^{R} and CTRL MGL samples. Focusing on common MECP2 occupation sites between KO^{R} and CTRL MGL samples (FIG. 2B), 58,521 sites were identified, the majority located in introns and intergenic regions (FIG. 2C). It was hypothesized that sites on promoter regions would predict potential MECP2 downstream targets that regulate microglial functions. Such promoter-transcription starting site (TSS) regions represent 4,069 (6.95%) of the total MECP2 sites found in both MGL samples (FIG. 2C). Among these, seven genes were shared in common with the targeted transcriptomics (FLNB, ITGAM, LAPTM5, PDGFRA, PIK3CG, PPARG, PTGS1), and 653 genes overlapped with the untargeted RNA-seq. Top canonical pathways obtained from genes in which MECP2 protein occupied promoter regions included several previously revealed pathways from transcriptomics, including integrin signaling, Fcγ receptor-mediated phagocytosis in macrophages and monocytes, TREM1 signaling, complement system, neuroinflammation signaling pathway and actin cytoskeleton signaling (FIG. 2D). From the catalog of promoter-TSS occupation sites, MECP2 occupied the promoter region of two microglial marker genes (P2RY12 and OLFML3), several chemokines, and their receptors (CXCL3, CCR5, CXCR2, CCR3, CCL13), and CYBB—a gene involved in the generation of reactive oxygen species (ROS). MECP2 also occupied the promoter region of genes that belong to the complement system (CD93, C1QA, C1QB. C1QTNF5, C1QTNF6, C2, C3, and C9) and to the immunoregulatory receptors related to microglial engulfment (SIGLEC5, SIGLEC7, and SIGLEC14). Further, MECP2 occupied the promoter of genes involved in the phagocytosis process (AXL, ITGAM, ITGA4, and MRC1) (FIG. 2E). Several known “binding” motifs were identified: CTCF⁶⁰ (Homer), BORIS (Zf) (GSE32465, Homer), PU.1 (ETS) (GSE21512, Homer), SpiB (ETS) (GSE56857, Homer), ELF5 (ETS) (GSE30407, Homer), but also de novo motifs: SpiB (ETS) (GSE56857, Homer), BORIS (Zf) (GSE32465, Homer), STAT5 (Stat) (GSE12346, Homer), PB0057.1Rxa_1 (Jaspar) and FOS::Jun (Jaspar) (FIG. 2F-G).

As another tool to complete the search for microglial cells pathways altered upon loss of MECP2, a label-free liquid chromatography-mass spectrometry (LC-MS) proteomic analysis was conducted on CTRL, KO and KO^{R} microglial cells (FIG. 2H-I, FIG. 8D-G), once again picking up pathways shared in common with targeted or bulk transcriptomics and as MECP2 ChIP-seq (FIG. 8D). There were 473 targets revealed by proteomics in which MECP2 was found to occupy their promoter, pointing to canonical pathways, including phagosome formation, integrin signaling, phagosome maturation, and actin cytoskeleton signaling pathways (FIG. 2H-I, FIG. 8D-G). It is noted several top causal networks having significant z-score alterations (activated or inhibited), including integrin, NCOR1, complement complex and pro-inflammatory cytokines, and chemokines (FIG. 8F). Top diseases and bio-functions pointed towards engulfment, cell movement, invasion, cytoplasm organization, and Alzheimer's disease annotations (FIG. 8E) with z-scores mostly predicting a decreased activation state. Both ITGAX and PTGS1 were found at the intersection of the proteomic and transcriptomic datasets (FIG. 8G). Collectively, our multi-omics results highlighted the phagocytosis, integrin signaling, and actin cytoskeleton signaling pathways as potentially disrupted due to the lack of MECP2 protein in microglia.

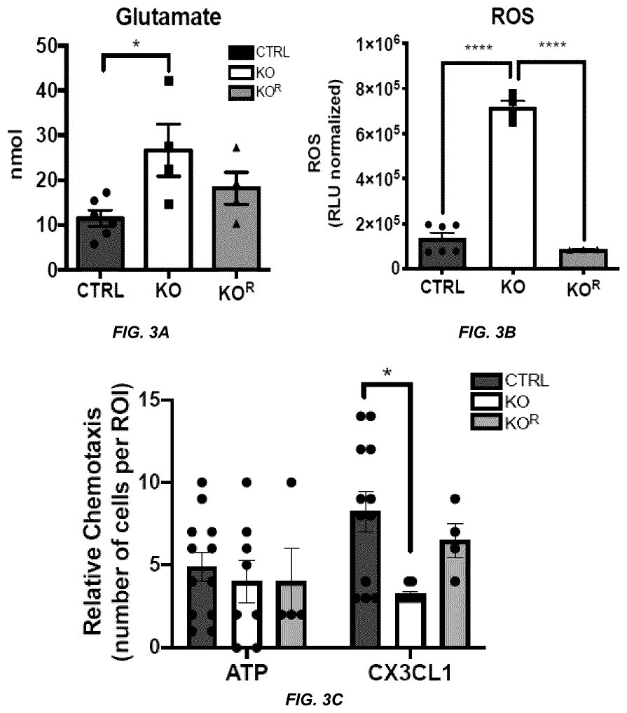

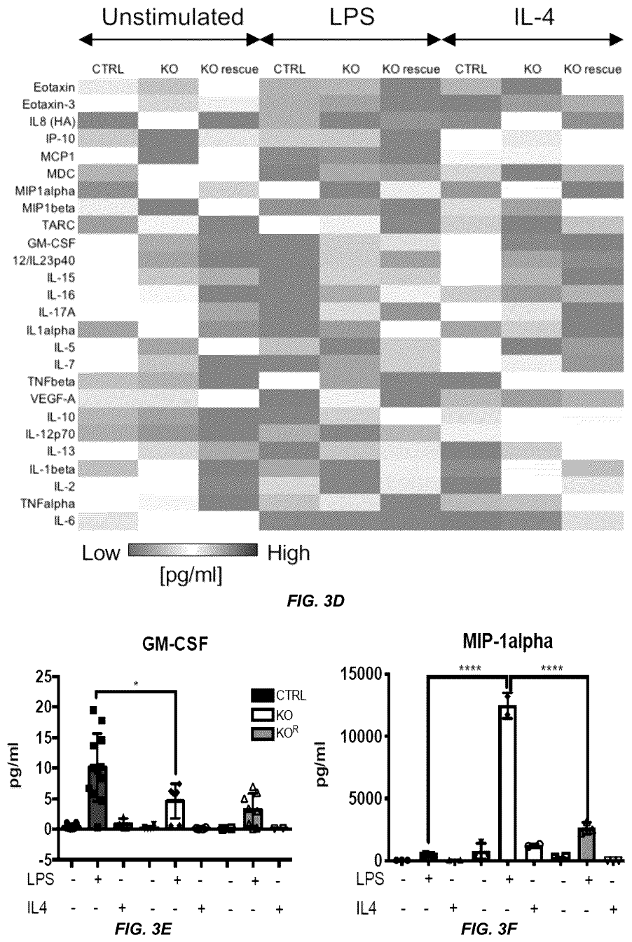

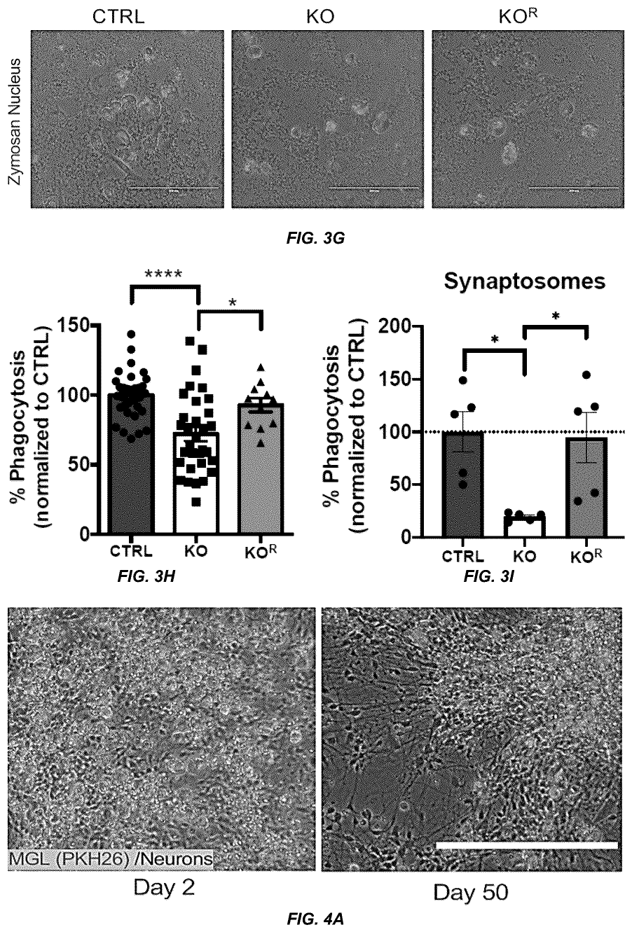

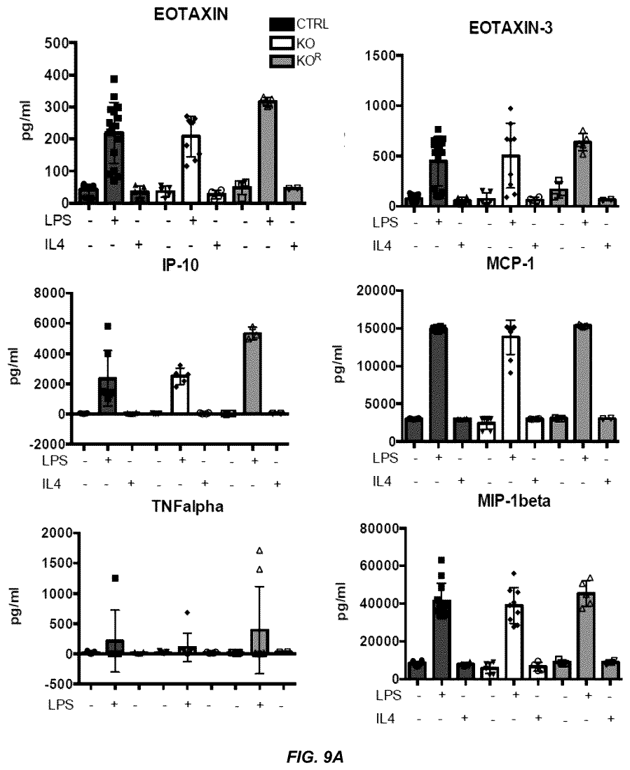

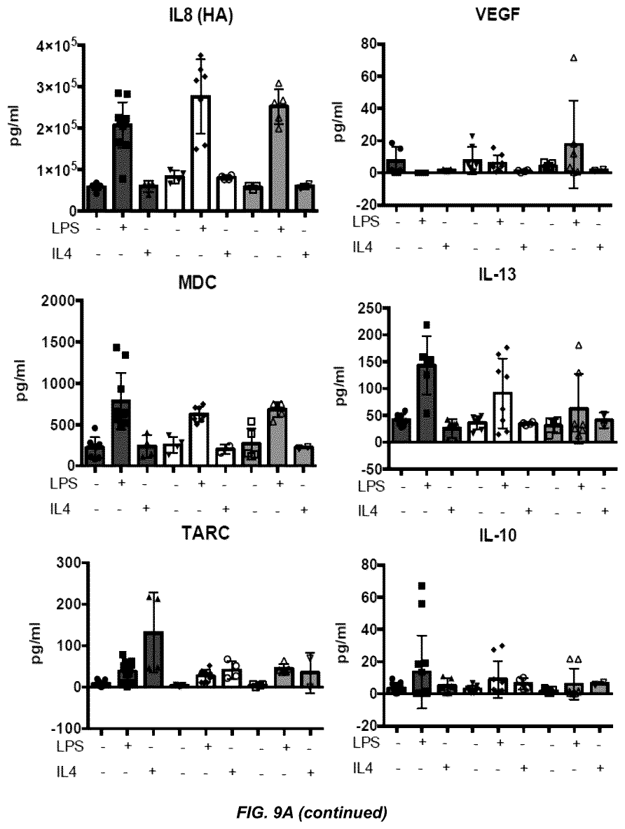

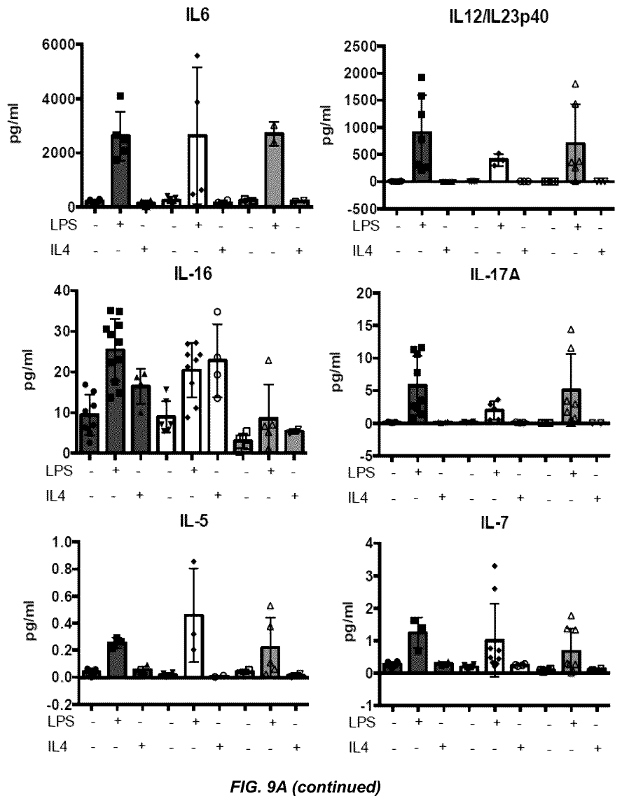

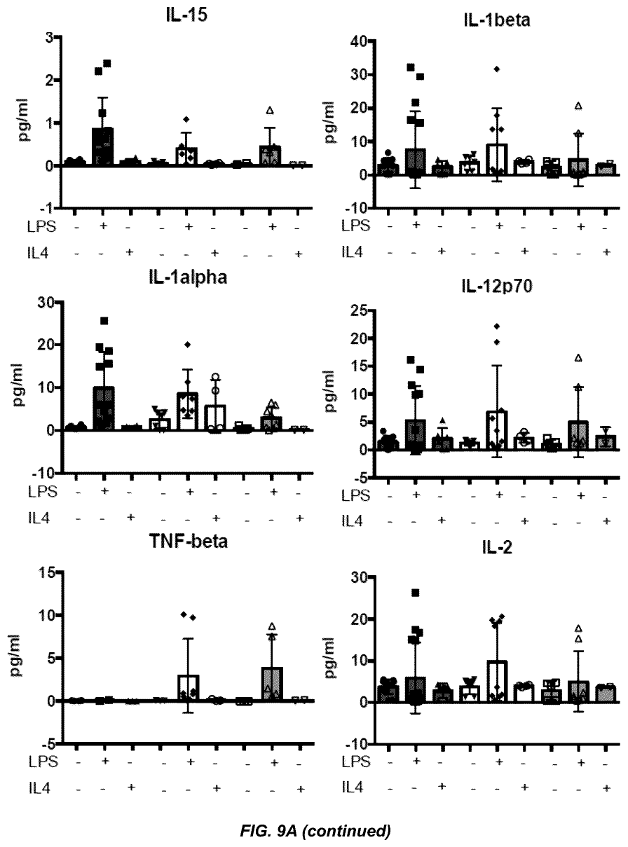

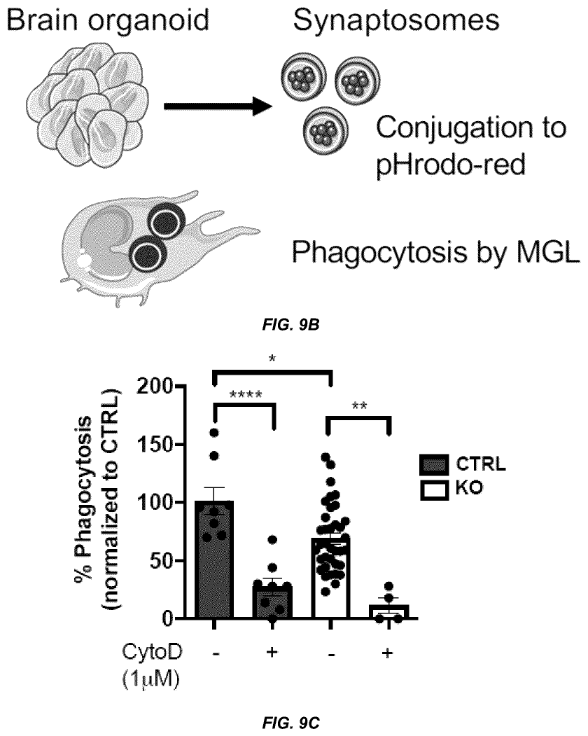

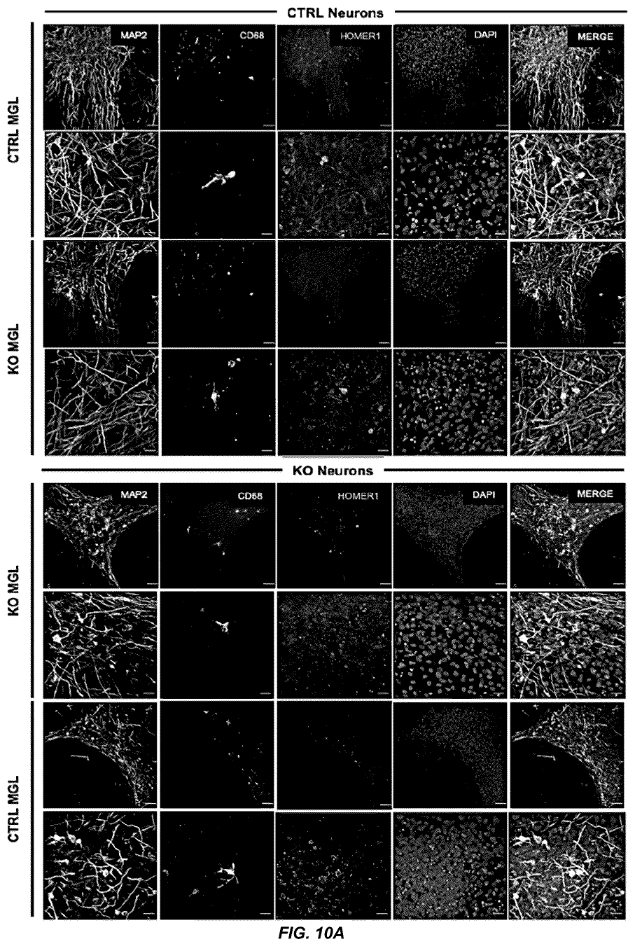

The absence of MECP2 leads to alterations in microglial functions. Next, analysis of microglial functions associated with the converging molecular pathways found in various datasets, such as cell movement, neuroinflammation, phagocytosis, was performed by first quantifying the release of several factors by MGL into the media. Glutamate has been postulated to be a neurotoxic factor released by microglial cells during diseased conditions. KO MGL released twice as much glutamate as CTRL or KO^{R} MGL (FIG. 3A). KO MGL also released three times more ROS compared to CTRL and KO^{R} MGL (FIG. 3B), validating the screening results of increased NADPH oxidase 4 (NOX4) gene expression and MECP2 ChIP-seq occupancy of the promoter of the CYBB gene encoding NADPH oxidase 2 (NOX2) (FIGS. 1I and 2E). Microglia sense and migrate towards chemoattractant molecules, and pathways associated with migration, cell invasion, and chemokine signaling were identified in transcriptomic analyses. While all three genotypes of microglia had similar chemotaxis in response to ATP, KO MGL had decreased chemotaxis in response to fractalkine (CX3CL1) (FIG. 3C), a neuronal chemoattractant that binds CX3CR1 expressed on microglial cells (FIG. 6B). Microglia respond to inflammatory stimuli by secreting cytokines and chemokines. Thus, levels of 26 cytokines and chemokines released by unstimulated MGL were measure, and those stimulated with the pro-inflammatory lipopolysaccharides (LPS) and anti-inflammatory IL-4 (FIG. 3D-F, FIG. 9A). Globally, all three genotypes, CTRL, KO, and KO^{R} MGL has similar levels of cytokine release into the media and responded similarly to both stimulations. Upon activation with LPS, the majority of pro-inflammatory cytokines release was increased. In contrast, the IL-4 stimulation had the opposite effect, suggesting that the inflammatory response to LPS or IL-4 is independent of MECP2 protein expression (FIG. 3D-F, FIG. 9A). Two exceptions between KO and healthy MGL were noted: LPS-stimulated KO MGL released significantly more MIP-1alpha and less GM-CSF compared to CTRL or KO^{R} MGL (FIG. 3D-F), consistent with altered MGL chemotaxis in the absence of MECP2 protein while the rest of the 26 analytes showed similar results, suggesting that MECP2 did not impact the overall immune response to LPS or IL4 in human MGL in the experiments.

Dysregulated phagosome formation, phagocytosis, phagosome maturation, integrin signaling, complement, and actin cytoskeleton signaling pathways were identified across all transcriptomic, ChIP-seq, and proteomic screens. Thus, experiments were performed on a microglial function: phagocytosis. CTRL and KO^{R} MGL had similar levels of phagocytic engulfment of zymosan particles, while KO MGL showed decreased phagocytosis (FIG. 3G-H). Upon treatment with Cytochalasin D to inhibit actin polymerization, the engulfment of zymosan particles was reduced, confirming a phagocytotic process (FIG. 9B-C). To confirm these data, pHrodo was used, a fluorescent indicator of cellular uptake to acid compartments and lysosomes, and conjugated human brain organoid-derived cellular fractions enriched in synaptosomes as the target of phagocytosis (FIG. 3I, FIG. 9B-C). CTRL and KO^{R} efficiently phagocytosed synaptosomes similarly, while KO MGL engulfed synaptosomes 80% less compared to CTRL and KO^{R} (FIG. 3I).

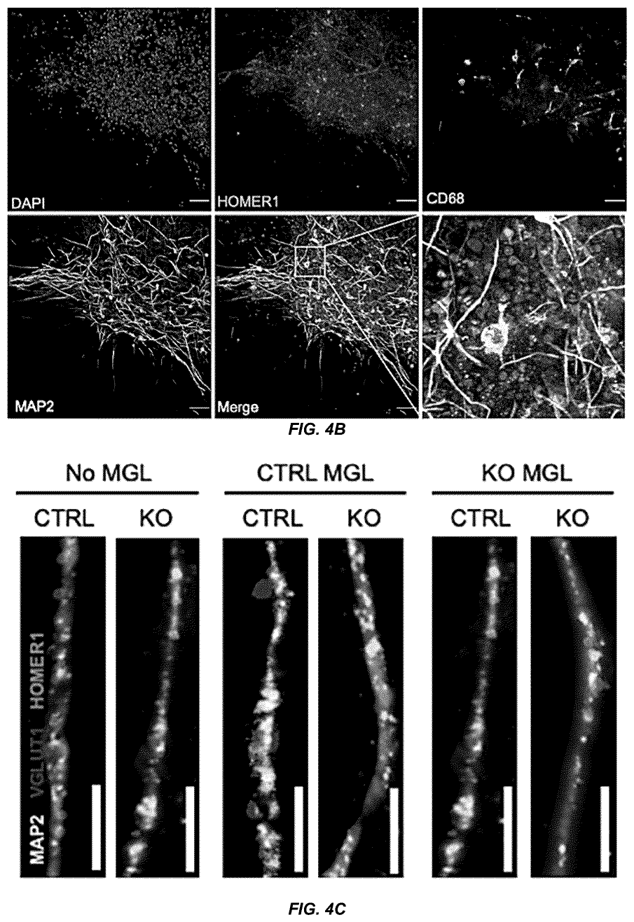

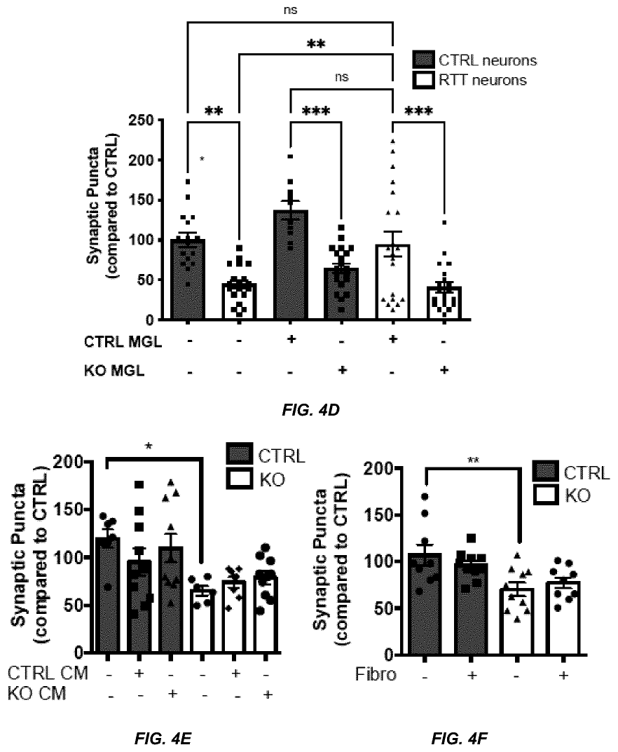

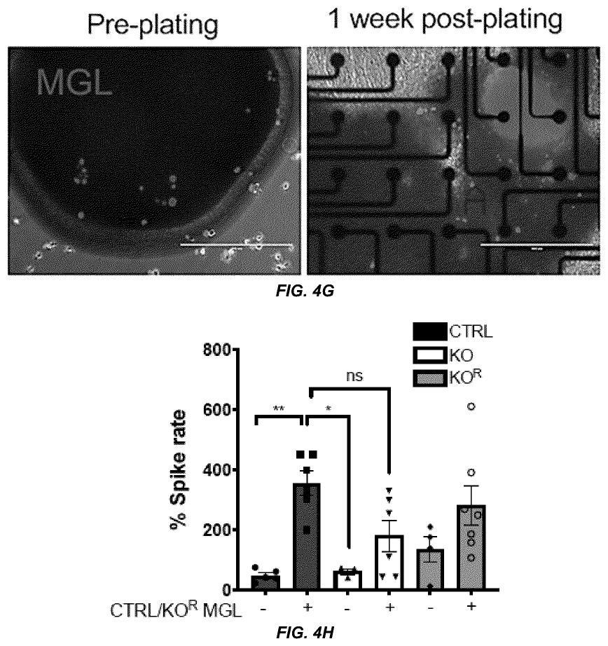

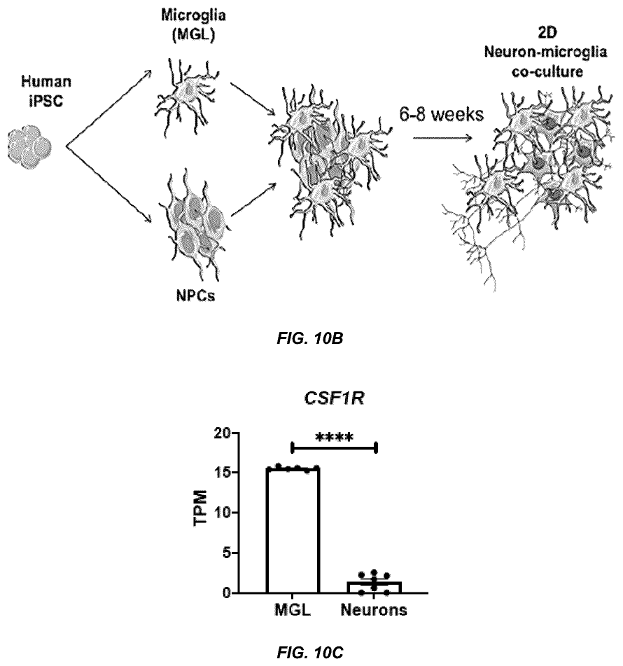

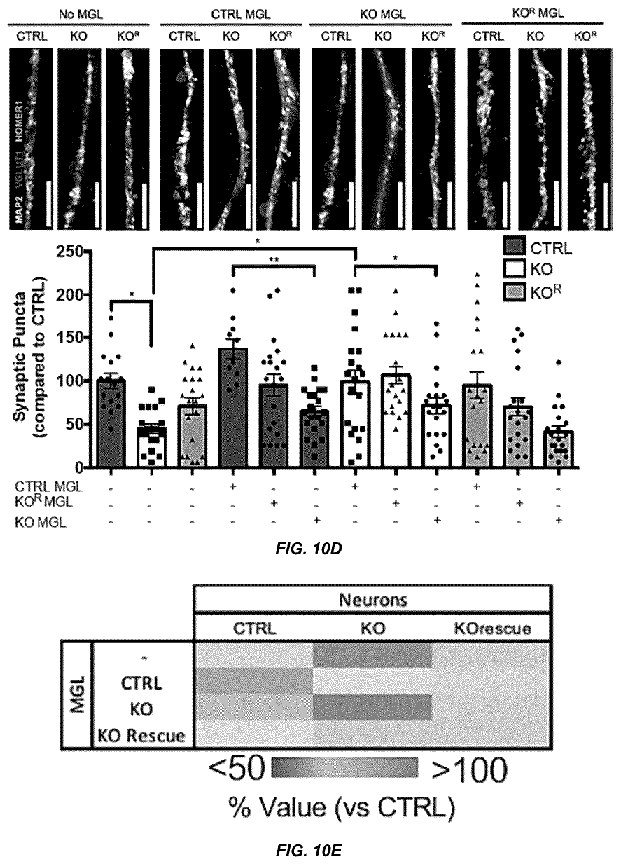

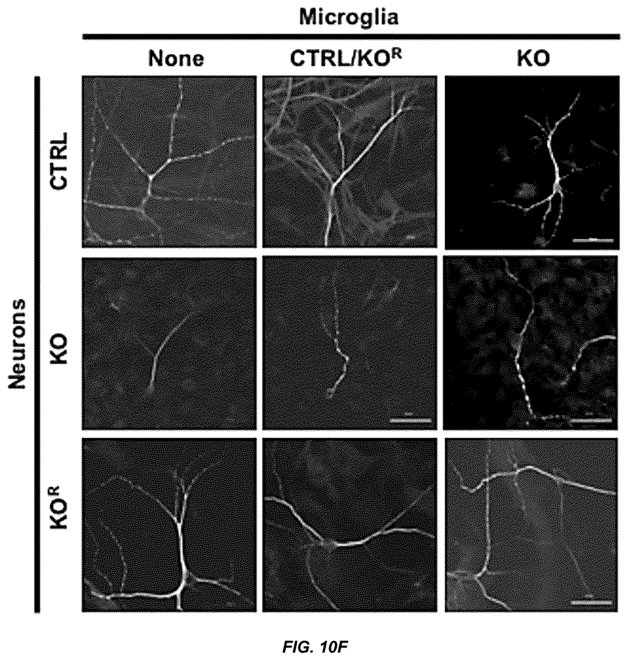

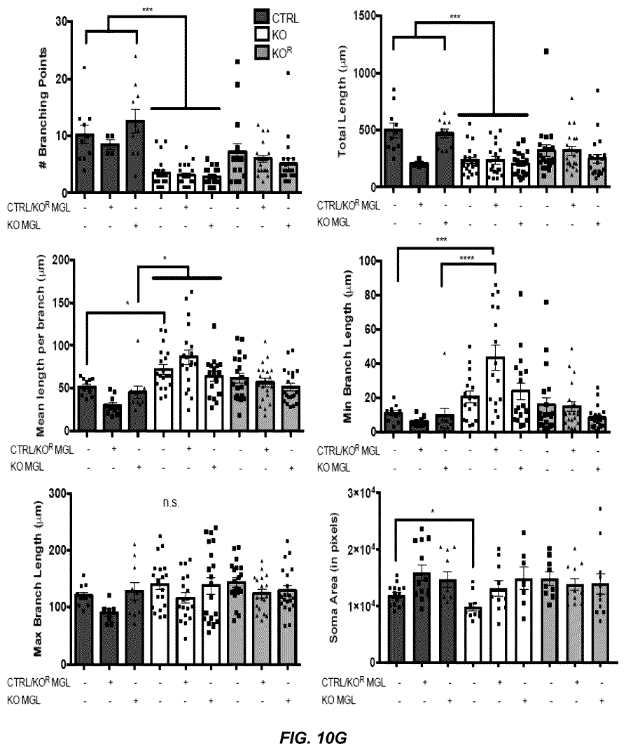

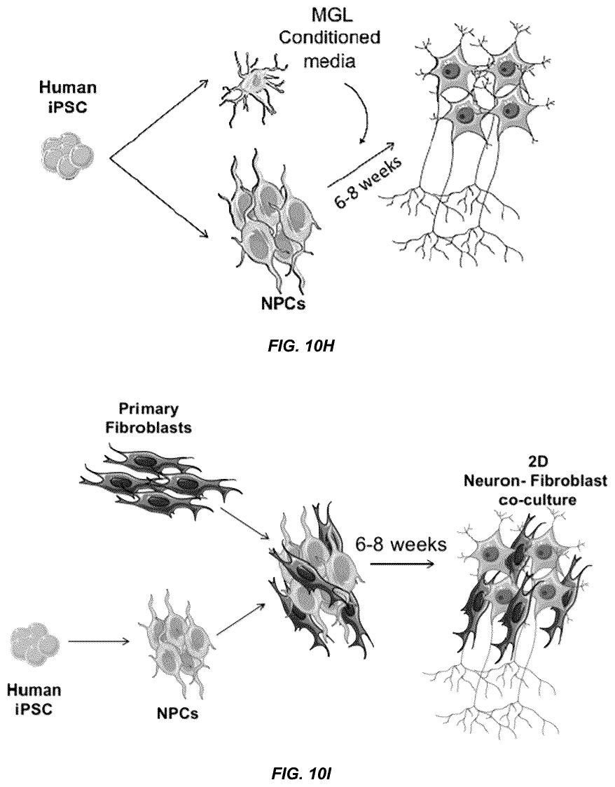

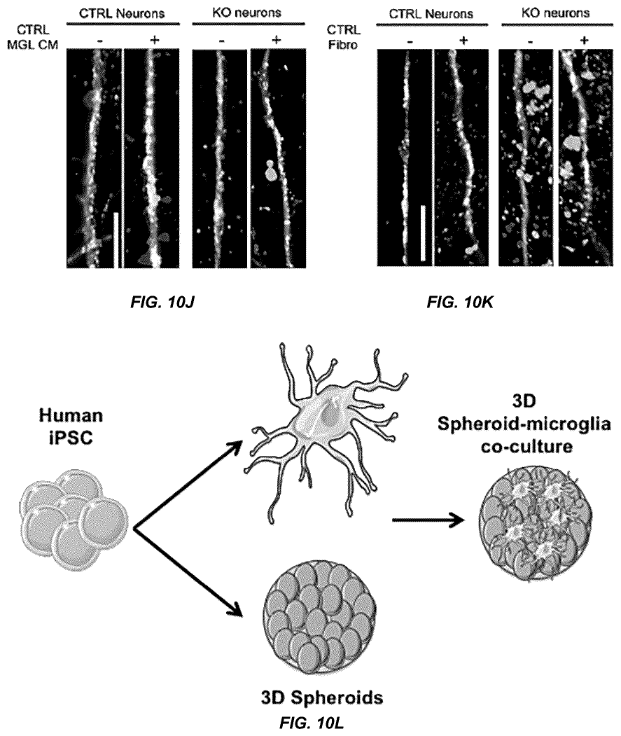

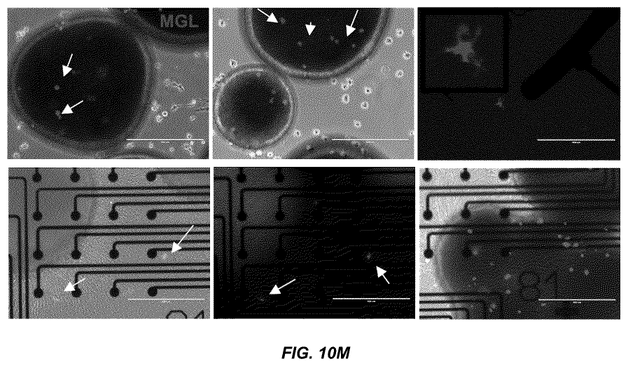

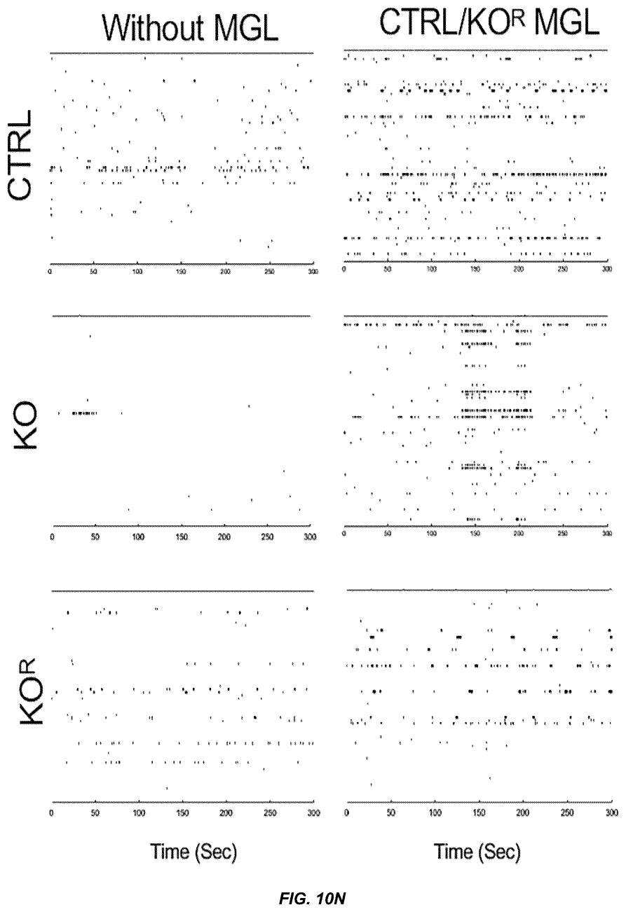

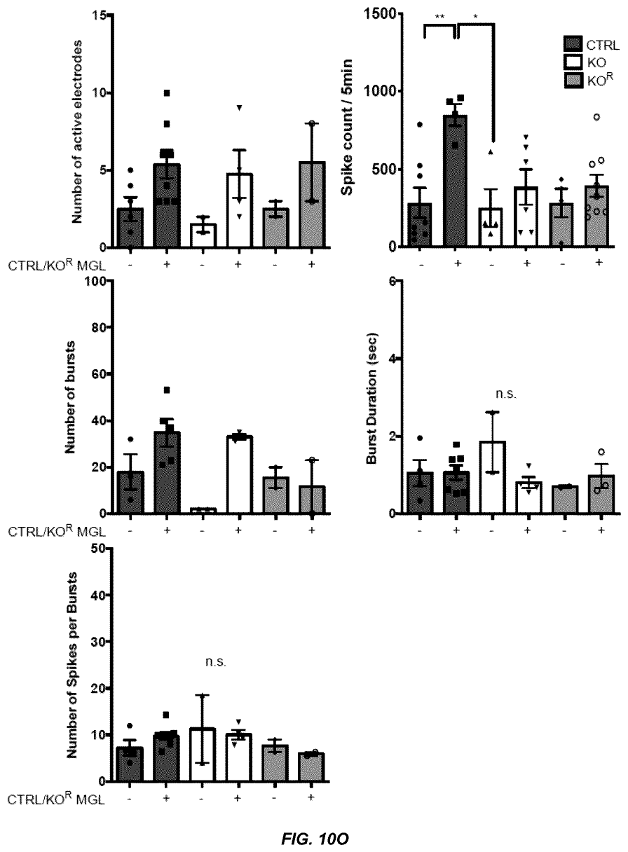

KO^{R}/CTRL MGL rescues the synaptic defects when co-cultured with KO neurons. Given that several key microglial functions, such as efficient phagocytosis were disrupted, it was hypothesized that these functional alterations could impact neuronal development and connectivity. To address this question, hiPSC-derived neuron-MGL co-culture experiments were performed by adding MGL onto neural progenitor cells (NPC) and allowed for cortical differentiation after retrieval of bFGF (FIG. 4, FIG. 10A-K). The MGL were pre-labeled with a fluorescent membrane dye, PKH26, suitable for long-term studies, and their presence in culture was monitored during neuronal differentiation for 6-8 weeks (FIG. 4A-B). As expected, the presence of IL-34 and MCSF in the media did not alter the synaptogenesis (FIG. 10D-E), as these ligands only bind to CSF1R which is highly expressed by MGL rather than neurons (FIG. 10C). After eight weeks of co-culture, the number of functional synapses defined as colocalization of pre- and post-synaptic proteins (=colocalized synaptic puncta, CSP) were counted (FIG. 4C-D, FIG. 10D-E). MECP2 KO neurons displayed 50% less CSP compared to CTRL and KO^{R} neurons (FIG. 4C-D, FIG. 10D-E). Adding CTRL MGL onto CTRL neurons significantly increased the number of CSP (FIG. 4C-D, FIG. 10A, B, D). Importantly, adding CTRL or KO^{R} MGL onto KO neurons rescued the synaptic defects, suggesting that MeCP2 re-expression in MGL is sufficient to rescue synaptogenesis (FIG. 4C-D, FIG. 10D-E). While adding CTRL/KO^{R} MGL onto KO neurons rescued the CSP, the number of branching points nor the neurite length changed (FIG. 10F-G). There was an increase in soma size of KO neurons upon addition of MGL (FIG. 10G). To determine whether MGL-induced synaptogenesis required physical contact of microglia with neurons or occurred through secreted factors by microglial cells, synaptogenesis assays were performed using CTRL or KO conditioned media from MGL (FIG. 4E, FIG. 10H and J). Adding CTRL or KO MGL conditioned media did not affect the CSP number in CTRL or KO neurons, suggesting that the effect of MGL on neurons is mediated by cell-to-cell interactions (FIG. 4E, FIG. 10H and J). As a control, the addition of healthy human primary fibroblasts did not affect synaptogenesis, suggesting the results observed on neurons are microglia-specific (FIG. 4F, FIG. 10I and K).

To assess if an increased synapse number translated into increased neuronal network activity, self-assembled 3D cortical spheroid-MGL co-cultures were performed (FIG. 4G-H, FIG. 10K-O), which showed that KO cortical spheroids had decreased synchronized neuronal activity. Cortical spheroids were co-cultured with MGL in suspension and then plated onto multi-electrode arrays (MEA), and the neural network activity was recorded after a week of co-culture (FIG. 4G-H, FIG. 10L-O). Adding CTRL MGL onto CTRL cortical spheroids significantly increased the spike rate and the number of spikes, in agreement with the observed increased number of CSP (FIG. 4G-H, FIG. 10L-O). KO cortical spheroids without MGL had lower spiking rate compared to CTRL spheroids with CTRL MGL, but adding CTRL or KO^{R} MGL increased their spiking rate, reverting this phenotype (FIG. 4G-H, FIG. 10L-O). However, adding CTRL or KO^{R} MGL onto CTRL, KO^{R}, or KO spheroids, did not change the burst duration, number of spikes per burst, number of active electrodes, or number of bursts (FIG. 100). Collectively, the data revealed that CTRL/KO^{R} MGL rescued the synaptic defects observed in cortical neurons lacking MeCP2, both at the level of the number of CSP and neuronal activity (FIG. 4G-H, FIG. 10L-O).

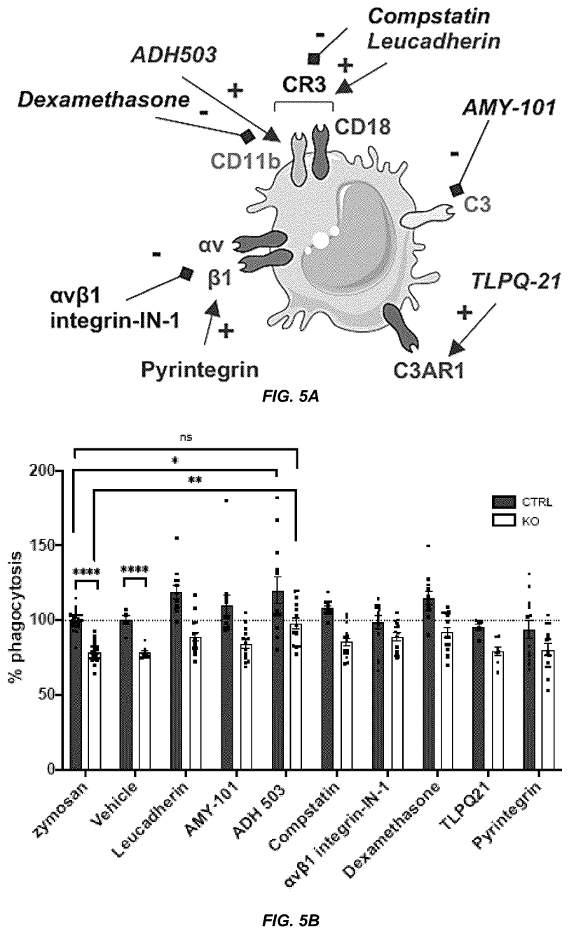

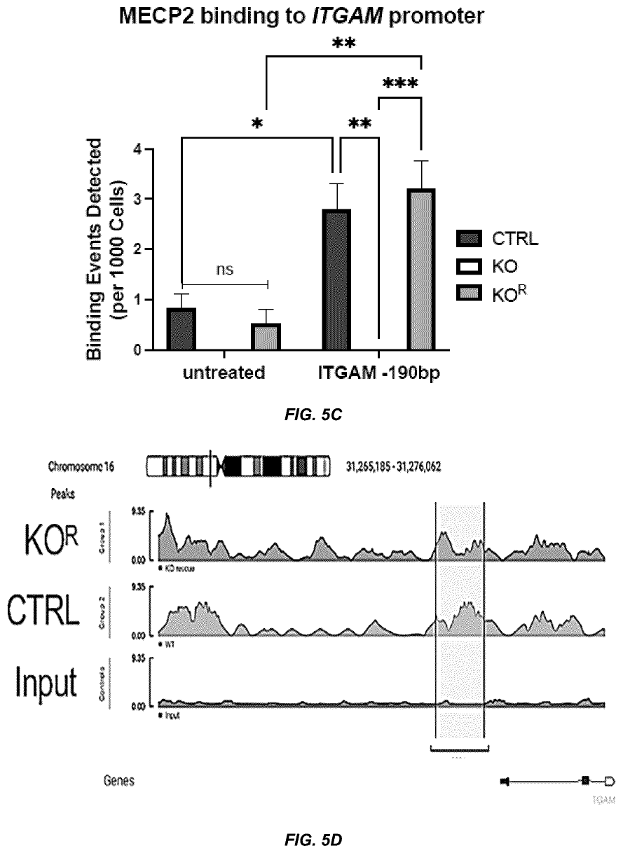

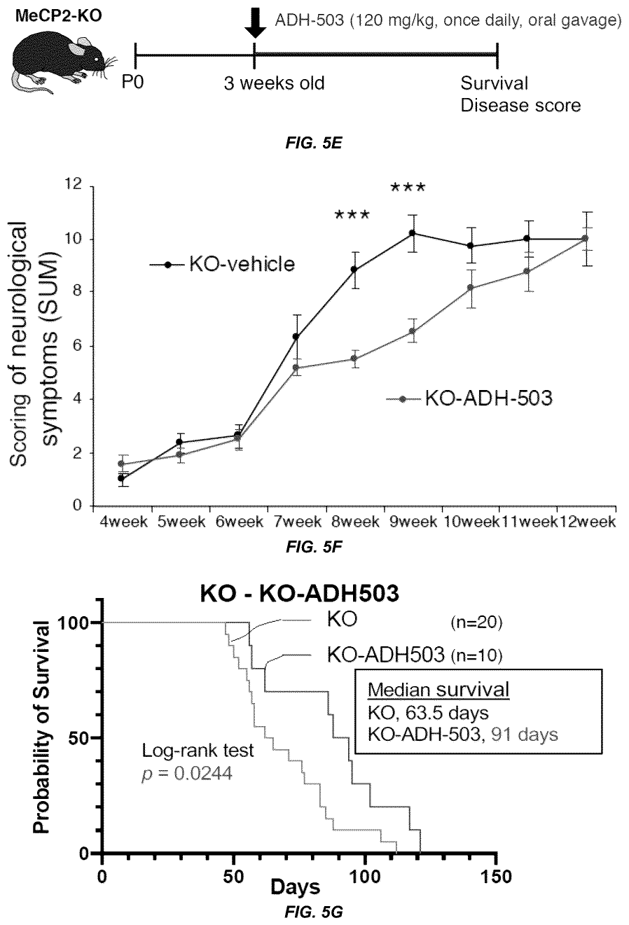

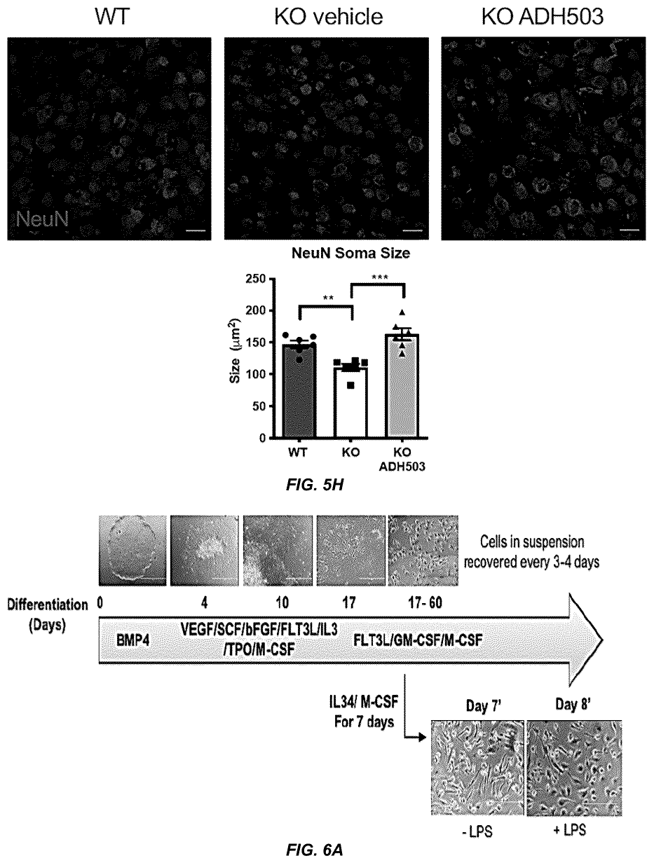

ADH-503 rescues phagocytosis in KO MGL, improves disease course and survival of MeCP2 KO mice. Given that the phagocytosis is an important microglial function, assay to screen for therapeutic compounds that could potentially rescue the phagocytosis defects and help understand better the mechanisms by which MECP2 controls the phagocytosis were used (FIG. 3H-I, FIG. 9C). 8 compounds were chosen that were either inhibitors or agonists of different integrin and complement system, that were identified (FIG. 2, FIG. 3 and FIG. 5A). Among these compounds, ADH-503, a CD11b agonist, for which the gene encoding for CD11b, ITGAM, is one of the targets for MECP2 occupancy (FIG. 2E, FIG. 5C-D), that was able to rescue the phagocytosis in KO MGL (FIG. 5B). Defects in microglial phagocytosis have been previously reported in MeCP2-KO mice. Therefore, MeCP2-KO mice were treated, starting at 3 weeks of age daily and by oral gavage, with 120 mg/kg ADH-503, a dose previously known to be safe (FIG. 5E). MeCP2-KO mice that received ADH-503 daily showed slowed disease progression (FIG. 5F) and, importantly survived 40% longer than the ones that received the vehicle treatment (FIG. 5G). Moreover, the MeCP2-KO mice that were treated with ADH-503 had their soma size rescued to the WT proportions (FIG. 5H), suggesting that ADH-503 had an impact on the CNS.

It will be understood that various modifications may be made without departing from the spirit and scope of this disclosure. Accordingly, other embodiments are within the scope of the following claims.

1. A method for treating a disease or disorder caused by abnormal neuronal development in a subject in need thereof, comprising administering to the subject a therapeutically effective amount(s) of an agonist of CD11b and/or genetically normal microglial cells.

2-3. (canceled)

4. The method of claim 1, wherein the subject is less than 25 years of age.

5. (canceled)

6. The method of claim 1, wherein the agonist is ADH-503 or other leukadherin-1 CD11b agonist.

7. The method of claim 1, wherein the genetically normal microglial cells are derived from induced pluripotent stem cells (iPSCs).

8. The method of claim 1, wherein the disease or disorder is Rett Syndrome.

9. The method of claim 1, wherein the disease or disorder is selected from the group consisting of amyotrophic lateral sclerosis (ALS), Alzheimer's disease (AD), schizophrenia, autism spectrum disorders, diffuse leukoencephalopathy with spheroid formation, and frontal Lobar degeneration (FTLD).

10. The method of claim 7, wherein the iPSCs are autologous to the subject.

11. The method of claim 7, wherein the iPSCs are from a Rett subject and wherein the IPSCs are genetically modified to express MECP2.

12. The method of claim 7, wherein the subject has a deficit in normal phagocytosis and wherein the iPSCs are treated with a CD11b agonist prior to and concurrently with differentiation to microglial cells.

13. A human-based neural drug screening platform for identifying therapeutic compounds that can ameliorate or rescue deleterious biological effect(s) resulting from abnormal expression or activity of genes in neuronal development disorders, comprising:
(a) a two- and three-dimensional coculture of neurons and microglial cells, wherein (i) the neurons, (ii) the microglial cells or (iii) the neurons and microglial cells, have been differentiated from human stem cells that have mutation(s) affecting normal neuronal development;
(b) contacting the coculture with a candidate agent; and
(c) evaluating whether the candidate drug rescues or ameliorates deleterious gene expression;
wherein if a candidate drug rescues and/or ameliorates one or more deleterious biological effects the candidate drug is a therapeutic compound.

14. The platform of claim 13, wherein the human stem cells are induced pluripotent stem cells (iPSCs).

15. The platform of claim 14, wherein the iPSCs are obtained from a subject having a genetic neurological disease or disorder.

16. The platform of claim 14, wherein the candidate test agent is a small molecule drug.

17. The platform of claim 16, wherein the small molecule drug effects CD11b expression and/or activity.

18. The platform of claim 17, wherein the small molecule drug mediates one or more activities selected from the group consisting of phagocytosis, cell adhesion and migration, restricts TLR and IFN-1 signaling, inhibits B cell activation, inhibit T cell activation, inhibits Th17 differentiation, suppresses CD maturation and function.

19. The platform of claim 16, wherein the small molecule agent promotes CD11b/CD18 dimerization to form CR3.

20. A co-culture of microglia cells and cortical spheroids, wherein the microglial and/or cortical spheroids were differentiated from a population of induced pluripotent stem cells (iPSCs).

21. The co-culture of claim 20, wherein the population of iPSCs are human iPSCs.

22. The co-culture of claim 20, wherein the population of iPSCs are obtained from a subject having a neurological disease or disorder.

23. The co-culture of claim 20, wherein the iPSCs are genetically engineered.

24-25. (canceled)

26. The co-culture of claim 20, wherein the microglial cells and cortical spheroids are genetically heterogenous.

27. A method for screening a therapeutic agent for treating a neurological disease or disorder, the method comprising:
(a) contacting a test agent with a culture of claim 20; and
(b) measuring a biological marker associated with the neurological disease or disorder.

28. The method of claim 27, wherein a beneficial change in the biological marker is indicated the test agent is capable of treating a neurological disease or disorder.

29. The method of claim 28, wherein biological marker is selected from the group consisting of phagocytosis, calcium signaling, amyloid beta phagocytic function, glutamate metabolism and any combination thereof.

30-31. (canceled)

32. The method of claim 27, wherein the neurodegenerative disease is Alzheimer's disease or multiple sclerosis.

33. (canceled)
